(19) United States
(12) Patent Application Publication
(10) Pub. No.: US 2023/0062964 A1
(43) Pub. Date: Mar. 2, 2023

(54) MODIFIED MICE THAT PRODUCE HEAVY CHAIN ONLY ANTIBODIES

(71) Applicants: New York University, New York, NY (US); Ingenious Targeting Laboratory, Inc., Ronkonkoma, NY (US)

(72) Inventors: Sergei Koralov, Millburn, NJ (US); Timothy Borbet, Brooklyn, NY (US); Paul Sheiffele, Mt. Sinai, NY (US); Wei Wang, Sayville, NY (US)

(21) Appl. No.: 17/823,506

(22) Filed: Aug. 30, 2022

Related U.S. Application Data
(60) Provisional application No. 63/238,703, filed on Aug. 30, 2021.

(51) Int. Cl. A01K 67/027

(52) U.S. Cl. CPC A01K 67/0278; A01K 67/0271; A01K 2207/15; A01K 2227/105; A01K 2217/052; A01K 2267/01

(57) ABSTRACT

Provided are compositions and methods for producing modified non-human animals that produce heavy chain only antibodies (HCAbs), and modified non-human animals produced by the compositions and methods, and isolated HCAbs produced by the HCAbs.

### CROSS REFERENCE TO RELATED APPLICATIONS

This application claims priority to U.S. provisional application No. 63/238,703, filed Aug. 30, 2021, the entire disclosure of which is incorporated herein by reference.

### SEQUENCE LISTING

The instant application contains a Sequence Listing which has been submitted in .xml format and is hereby incorporated by reference in its entirety. Said .xml copy was created on Aug. 30, 2022, is named “058636_00549_ST26.xml”, and is 123,731 bytes in size.

### FIELD OF THE DISCLOSURE

The present disclosure is related to compositions and methods for producing modified non-human animals that produce heavy chain only antibodies (HCAbs), and modified non-human animals produced by the compositions and methods, and isolated HCAbs produced by the HCAbs.

### BACKGROUND

Antibodies, the pathogen-neutralizing proteins produced by the immune cells, are critical for the humoral immune response against bacteria, viruses and cancer. While playing this important role in immune defense, they also have vast applications for therapeutics, diagnostics and research.

Conventional antibodies (Abs) comprise two heavy and two light immunoglobulin protein chains. By contrast, Heavy-Chain only antibodies (HCAbs), found alongside conventional Abs in camelid and shark species, lack an immunoglobulin light chain. Despite their scarcity in nature, HCAbs have favorable characteristics, including small size, high hydrophilicity, and conformational stability under environmental stresses that make them better suited for development of therapeutic applications than conventional Abs. Furthermore, the antigen binding single-domain fragments of these HCAbs, nanobodies, can be isolated, purified, and linked to other molecules to function as therapeutic agents or diagnostic sensors. Immunization of camelids to obtain HCAbs presents logistic and financial challenges, and in vitro technologies cannot fully recapitulate the natural selection that occurs in animals. In vitro single-chain Ab libraries also struggle with complex, membrane associated antigens that are often central targets for research and medicine. Thus, there is an ongoing and unmet need for new approaches to produce HCAbs. The present disclosure is pertinent to this need.

### BRIEF SUMMARY

The disclosure provides compositions and methods for making modified non-human animals that that produce partially humanized heavy chain only antibodies (HCAbs). Methods of producing HCAbs using the modified non-human animals are also included by the disclosure, as are HCAbs that are isolated from the non-human animals.

In one aspect, the disclosure provides a modified non-human animal, wherein the genome of the animal comprises: at least one unarranged immunoglobulin heavy chain variable domain, at least one unarranged immunoglobulin heavy chain D domain, and one unarranged immunoglobulin heavy chain J domain are operably linked to a functional non-human immunoglobulin heavy chain constant gene sequence, wherein the CH1 domain from constant region is deleted, enabling expression of single immunoglobulin heavy chain on a B cell. This type of modified non-human animal is referred to herein as Model I.

In another aspect the disclosure provides a modified non-human animal, wherein the genome of the animal comprises: at least one un-rearranged murine or camelid VHH, at least one human immunoglobulin heavy chain D domain, and one human immunoglobulin heavy chain J domain are operably linked to a functional non-human immunoglobulin heavy chain constant gene sequence. This type of modified non-human animal is referred to herein as Model III. In an embodiment, Model III comprises at least one human DH AND one human JH segment.

In one embodiment the modified non-human animal is a mouse.

The disclosure includes immunizing the described modified non-human animals so that the modified non-human animals produce HCAbs. The HCAbs can be separated from the modified non-human animals and sequenced to determine one or more complementarity-determining regions (CDRs). In embodiments, the disclosure includes determining at least CDR3 of the HCAbs. The disclosure further provides for recombinantly producing and using the HCAbs for a variety of purposes that will be apparent to those skilled in the art when given the benefit of the present description.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1 shows a representative approach for replacing mouse heavy chain locus with VHH and human D and J with a deletion of IgM, IgD, and CH1 domains. The representation in the figure is to the scale as shown by the scale bar on the left-bottom side of the figure. The top panel shows the general organization of endogenous mouse sequence covering heavy chain Ds, Js, IgM, IgD, and constant region IgG3 including Ch1 domain in its native structure. The top panel also include the adjacent 5′ up-stream and 3′ down-stream mouse sequences on mouse chromosome 12. The middle panel depicts the targeting vector used to replace the mouse heavy chain region. The targeting vector has 16 segments: DNA seg. 1: A 5′ mouse homology arm; DNA seg. 2: A prokaryotic selection cassette with a loxP recombinant site, which can serve as a further deletion or additional DNA insertion landing pad; DNA seg. 3: A half hygromycine selection cassette and a Rox recombinant site to facilitate additional DNA landing; DNA seg. 4: A human IGHV 3-30 promoter DNA sequence; DNA seg. 5: A VHH from Llama 2 (DNA SEQ ID NO: 4); DNA seg. 6: A human IGHV 1-3 promoter DNA sequence; DNA seg. 7: A VHH from alpaca 2 (DNA SEQ ID NO: 2); DNA seg. 8: A human IGHV 1-2 promoter DNA sequence; DNA seg. 9: A VHH from llama 1 (DNA SEQ ID NO: 3); DNA seg. 10: A human IGHV 6-1 promoter DNA sequence; DNA seg. 11: A VHH from camel 1 (DNA SEQ ID NO: 1); DNA seg. 12: A human DNA sequence connecting the 3′ end of human IGHV 6-1 to the first human heavy chain D (D1-1); DNA seg. 13: A human DNA sequence comprising complete human heavy chain Ds; DNA seg. 14: A human DNA sequence comprising complete human heavy chain Js; DNA seg. 15: A pro/eukaryotic G418/Neo selection cassette; DNA seg. 16: A 3′ mouse homology arm including mouse constant region C4 (from exon 1 to exon 4). The exons are represented by vertical bars. The bottom panel depicts the targeted event. In the bottom of the figure, “targeted” means the targeting vector with VHHs and human sequence replaced mouse heavy chain Ds, Js, IgM, IgD, and CH1 at the mouse endogenous locus on chromosome 12.

FIG. 2 panels show a flow cytometry analysis of mesenteric lymph nodes of 6 week old WT and homozygous VHH-human D and J mice. The analysis includes evaluation of IgG3, IgM, and Igκ light chain cell surface expression and the recruitment of conventional and HCAb expressing B cells into germinal centers (GC). Gated on B220+ CD19+ B cells, there were a dramatic difference between WT and homozygous VHH-human D and J mice. FIG. 2 panel A shows that the majority of B cells (gated on B220+ CD19+) of WT mice express IgM (88.6%). Only 0.35% are IgG3 positive. In sharp contrast, the majority (96%) of B cells in VHH-human D and J mice are IgG3 positive (gated on B220+ CD19+). This indicates that homozygous VHH-human D and J mice were able to use unarranged V, D, and J elements to generate single-chain antibody receptors on B cells after VDJ recombination. In addition, FIG. 2 panel B further demonstrates the single-chain expression of B cells because Igκ light chain expression was absent in homozygous VHH-human D and J mice (model III) on the plot, yet highly expressed in WT mice. A fluorescence minus one (FMO) control for Igκ light chain is shown as a control for a light chain deficient B cell population. FIG. 2 panel C shows that single-chain B cells responded to antigen stimulation in vivo indicated by the highlighted population of Fas^{high}, CD38^{neg} germinal center B cells.

FIG. 3 provides a summary in Table 1 of Junctional nucleic acid sequences for VHHs and human D and J targeting vector.

FIG. 4 provides a summary in Table 2 of origin and sequence description of VHH (Camel, Alpaca, and Llama).

FIG. 5 provides a summary in Table 3 of antigens and their sources.

FIG. 6 provides results in Table 4 of Elisa analysis from homozygous single chain mice challenged by human ApoE antigen (O.D.).

FIG. 7 provides results in Table 5 of Elisa analysis from homozygous single chain mice challenged by human Abeta Antigen (O.D.).

FIG. 8 provides results in Table 6 of Elisa analysis from homozygous single chain mice challenged by human IgM Antigen (O.D.).

FIG. 9 provides results in Table 7 of Elisa analysis from homozygous single chain mice challenged by Omicron Surface Antigen (O.D.).

FIG. 10 provides a summary in Table 8 of primers for detecting RNA expression of VHH and human D and J (PCR round 1).

FIG. 11 provides a summary in Table 9 of primers for detecting RNA expression of VHH and human D and J (PCR round 2).

FIG. 12 provides a summary in Table 10 of AP2-Human D and J based CDR3 of Humanized (human Ds and Js) HVV in the spleen.

FIG. 13 provides a summary in Table 11 of CA1-Human D and J based CDR3 of Humanized (human Ds and Js) HVV in the spleen.

FIG. 14 provides a summary in Table 12 of LA1-Human D and J based CDR3 of Humanized (human Ds and Js) HVV in the spleen.

FIG. 15 provides a summary in Table 13 of LA2-Human D and J based CDR3 of Humanized (human Ds and Js) HVV in the spleen.

FIG. 16 provides a summary in Table 14 of AP2-Human D and J based CDR3 of Humanized (human Ds and Js) HVV in the blood.

FIG. 17 provides a summary in Table 15 of LA2-Human D and J based CDR3 of Humanized (human Ds and Js) HVV in the blood.

FIG. 18 provides the sequences that are part of this disclosure with their corresponding sequence identifiers.

FIG. 19 provides a representative and non-limiting schematic of a modification according to Model I of this disclosure. Targeting of the murine IgH locus using two guides and CRISPR-Cas9 resulted in the removal of IgM and IgD exons and truncation of IgG3 by removal of exon 1 which contains the Ighg3 CH1 domain. Abbreviations: VH: variable heavy domain gene segments; DH: heavy diversity gene segments; JH: heavy joining gene segments; Eμ: IgM-related enhancer; Sμ, Sγ3, Sα: IgM-, IgG3-, IgA-related switch regions, Cμ, Cδ, Cγ3, Cα: heavy constant gene regions (not all shown); NEO: neomycin cassette; 3′RR: heavy chain regulatory region; HCAb: heavy-chain only antibody.

FIG. 20 provides a representative and non-limiting schematic of a modification according to Model III of this disclosure. BAC insertion into the endogenous murine IgH locus resulted in the removal of IgM and IgD exons, truncation of IgG3 by removal of exon 1 which contains the Ighg3 CH1 domain. Abbreviations: VHH: camelid variable heavy domain gene segments; hDH: human heavy diversity gene segments; hJH: human heavy joining gene segments; hEμ: human IgM-related enhancer.

FIG. 21. Flow cytometric analysis of spleen of Model I animals. The spleens of 6-week-old WT, heterozygous and Model I mice were evaluated for B cell IgG3, IgM and Igκ light chain expression using standard flow cytometry staining on a BD Fortessa. Panel A. Events shown are gated on B220+ CD19+ B cells and IgG3 and IgM expression is shown and reveals that Model I animals make exclusively IgG3 isotype antibodies with fewer than 1% (0.44%) of IgM cells present. By contrast, WT littermates have 89.6% IgM expressing B cells. Panel B. Gated on all B cells, Igκ expression is shown for a representative mouse of each genotype. No Igλ, light chain was detected in B cells form Model I animals (data not shown) and was thus not stained for in these experiments. This analysis highlights that a majority of B cells in Model I animals produce single-chain HCAbs of the IgG3 isotype.

FIG. 22. Single-cell analysis of V(D)J repertoire of Model I. FACS sorted naïve mature B cells (DAPI− CD43− CD19+B220+) were prepared for emulsions via 10×Controller and 10× Chromium 5′ libraries were prepared. Panel A. Circos plots showing repertoire of VH-JH associations highlights diverse repertoire of 100 s of immunoglobulin rearrangements for each genotype (WT and Model I homozygous B cells) with each unique variable region represented by a different bar on the outside rim. Panel B. IgG3 usage is evident in transcripts from Model I animals, while only infrequently being detected in WT animals (endogenous IgG3 usage in naïve C57BL/6 animals is ˜1%).

FIG. 23. Analysis of germinal centers in Model I animals: Analysis of CD19+ B220+ B cells from the spleens of the animals following immunization with SRBCs. The representative plots indicate that homozygous Model I animals can form germinal centers to an equal frequency as WT animals. Germinal centers are essential for high affinity antibody generation.

FIG. 24. Robust plasma cell differentiation in Model I animals: Ex vivo differentiation of CD43− naïve mature splenic B cells using LPS (10 ug/mL) stimulation for 72 hrs leads to robust differentiation of plasma cells in Model I animals. Proliferation dye staining indicates that Model I B cells are dividing fewer times to differentiate to plasma cells when compared to wild-type B cells.

FIG. 25. Model I mice generate a robust Ab response to SARS-CoV-2 Spike protein: Analysis of serological immunization of Model I HCAb mice via ELISA to SP2 configuration of Spike protein of SARS-CoV-2 following immunization regimen detailed on the left part of the figure. Incomplete Freud's adjuvant (ICA) was used to stimulate robust response following 2 immunizations with 5 ug of SP2 emulsified in ICA. Serum was collected 1 week after the second challenge and subjected to an ELISA for spike-protein specific-IgG.

FIG. 26. Antibody-target protein interaction measurement for sera from immunized Model I animals: Bio-layer interferometry revealed favorable affinity measurements for polyclonal sera following immunization of Model I with SP2 as outlined in FIG. 6. Association and dissociation constants were evaluated via Octet bio-layer interferometry using synthetic SP2 peptide as the target protein. A K_{D} in the nanomolar range was calculated for the heavy-chain only antibodies from Model 1 mice an indication of high-affinity binding.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every DNA and RNA sequence encoding polypeptides disclosed herein is encompassed by this disclosure. Amino acids of all protein sequences and all polynucleotide sequences encoding them are also included, including but not limited to sequences included by way of sequence alignments. Sequences of from 80.00%-99.99% identical to any sequence (amino acids and nucleotide sequences) of this disclosure are included. Such changes can comprise conservative or non-conservative amino acid substitutions, insertions, and deletions, and the like, provided the different polynucleotides and proteins retain their intended function. In some embodiments, conservative amino acid substitutions can be in the V region, D region, J region, C region, and Complementarity-determining regions (CDRs).

The disclosure includes all polynucleotide and all amino acid sequences that are identified herein by way of a database entry. Such sequences are incorporated herein as they exist in the database on the effective filing date of this application or patent. Camelid VHHs from alpaca, llama and camel were used in certain targeting constructs. The sequences encoding the VHHs were modified for codon optimization from original camelid germline elements (AP2, LA1, LA2, CA1).

The present disclosure provides compositions and methods for making modified mammals that can produce HCAbs. The modified mammals made according to the described methods are included in the disclosure. Methods of producing HCAbs using the modified mammals are also included by the disclosure, as are HCAbs that are isolated from the modified mammals. All variable sequences and each CDR present in the HCAbs are included in the invention. The disclosure includes all compositions of matter that are used to produce the modified mammals, e.g., all vectors, including but not necessarily limited to targeting vectors, linear dsDNA templates, and the like. The disclosure includes all cells that are used to make the modified mammals, including all types of modified stem cells. In embodiments, the modified cells are embryonic stem cells. In embodiments, the cells are induced stem cells. In embodiments, the disclosure includes modified non-human blastocysts and modified non-human embryos. In embodiments, modified embryonic stem cells produced according to the methods of this disclosure may be diploid (2n), or may be present a tetraploid (4n) mouse blastocyst. The disclosure further comprises pseudopregnant non-human mammals comprising the described modified embryonic stem cells, such as within a blastocyst.

The disclosure includes parental modified non-human mammals and their progeny. In embodiments, the modified mammals are any member of the order Rodentia including but not limited to mice, rats and rabbits. In embodiments, the modified mammals are Mus musculus.

As used herein. “a,” “an,” or “the” can mean one or more than one. As used herein, unless specifically indicated otherwise, the word “or” is used in the inclusive sense of “and/or” and not the exclusive sense of “either/or.”

As used herein, the term “homologous recombination” or “HR” refers to the natural, cellular process in which a double-stranded DNA-break is repaired using a homologous DNA sequence as the repair template (see, e.g., Cahill et al. (2006), From. Biosci. 11:1958-1976). The homologous DNA sequence may be an endogenous chromosomal sequence or an exogenous nucleic acid that was delivered to the cell.

As used herein, the term “BCR” means B cell receptor. A “human BCR” or B cell receptor” or “humanized BCR” refers to a partial or entire BCR that includes a human sequence.

Gene names of V and J regions are designated according to the ImMunoGeneTic (IMGT) nomenclature for B cell receptor of human or mouse.

The term “C region” and grammatical forms of it used herein refer to a constant region of an antibody heavy chain. The constant region domain has separate gene segments for hinge, transmembrane, and cytoplasmic regions, which provide signal transduction after binding an antigen by the immunoglobulin heavy receptor.

One skilled in the art will recognize that gene targeting is based on homologous recombination that occur between the targeting construct and the targeted endogenous sequence through homology arms, two sequences on the targeting vector that are homologous to endogenous DNA sequences.

The term “operably linked” refers to at least two genetic or protein elements that are joined together in a manner that enables them to carry out their intended function. In this disclosure, portions of humanized protein may be operably linked to retain proper folding, processing, transporting, expression, and other functional properties of the protein in the cell. Further, a nucleic acid sequence encoding a protein may be operably linked to DNA sequences (e.g., promoter, enhancer, silencer, insulator etc.) to retain proper transcription.

The term “replacement” refers to a process comprises of placing exogenous genetic material at an endogenous gene locus, As demonstrated in the Examples below, nucleotide sequences of endogenous non-human antibody B cell receptor heavy chain variable gene loci were replaced by nucleotide sequences corresponding to camelidae B cell receptor heavy chain variable gene loci.

Camelidae or camelids comprises at least camel, llama, alpaca, vicunas, and guanacos. As an alternative to the foregoing, variable regions from sharks may be used. The immunoglobulin heavy receptor locus refers to the genomic DNA comprising the B cell receptor heavy chain coding region including unrearranged V(D)J sequences, enhancer, silencer, insulator, constant domain sequence(s), and upstream or downstream (e.g., 5′ and3′ UTR, regulatory regions, etc.), or intervening DNA sequence (e.g., introns, etc.), or RNA with regulatory functions.

V(D)J recombination refers to mechanisms that contribute to the diversity of B cell receptor heavy chain in the vertebrate immune system. In vivo and in vitro systems of generating xenobiotic or hybrid heavy chain antibodies are provided, wherein rodent-derived cells produce single-chain antibodies utilizing a modified constant region and variable regions comprised of either murine, camelid, or human variable regions. The antibodies produced are independent of light chain pairing and many are directly humanized by virtue of utilizing human DH and JH elements.

### DETAILED DESCRIPTION OF THE INVENTION

Ever since the discovery of hybridoma technology by Kohler and Milstein in 1975 monoclonal antibodies have represented very powerful tools to explore biological processes. Although classical monoclonal antibodies and their Fab fragment or single-chain variable fragments (scFvs) derivatives remain most widely used, camelid-derived single-domain antibodies (sdAbs) have emerged as a credible next generation of antibodies offering several advantages over classic antibodies owing to their peculiar properties, including their small size, robust behavior, high affinity and specificity, and deep tissue penetration (Debie. J Control Release. 2020; 317: 34-42. Jovcevska. BioDrugs. 2020; 34: 11-26. Khodabakhsh. Int Rev Immunol. 2018; 37: 316-22.). Besides camelids, sdAbs can also be found in certain sharks (Konning. Curr Opin Struct Biol. 2017; 45: 10-6.). However, many previously available recombinantly produced single-chain antibodies were generated in large animals and sequences of these single chains were not of human origin, which limited their use in human therapy. With ever growing demand of human therapy, a camelid based single-chain antibody may cause Immunogenicity and other undesirable side effects in humans. This disclosure therefore in certain embodiments provides a non-human modified animal comprising in its genome a VHH variable region from a camelid, a human D, a human J, and operably linked to endogenous non-human constant region. In this camelid-human non-human animal, a majority of CDR3s comprise human D and J regions.

In some embodiments, genetically-modified non-human animals are provided that comprise a modified endogenous antibody heavy chain of B cell locus that comprises an exogenous sequence (e.g., a human sequence and a camelids sequence) through homologous recombination (HR) in mouse ES cells. The animals are generally able to pass the modification to progeny, i.e., through germline transmission.

In some embodiments, the modified mammal of this disclosure is a mouse of a C57BL strain selected from C57BL/6, C57BL/6ByJ, C57BL/6J, C57BL/10, C57BL/6NJ, C57BL/6NIH, B6NTac, C57BL/A, C57BL/KaLwN, C57BL/GrFa, C57BL/10Cr, C57BL/10ScSn, C57BL/An, and C57BL/Ola.

In some embodiments, the mouse is selected from the group of 129 strain comprising of 129P1, 129P2, 129P3, 129X1, 129S1, 12951/SV, 12951/SvIm, 129S2, 129S4, 129S5, 12959/SvEvH, 129S6, 129/SvEvTac, 129S7, 129S8, 129T1, 129T2. In some embodiments, the mouse is a BALB strain, e.g., BALB/c strain. In some embodiments, the mouse is a mix of a BALB strain and another strain. In some embodiments, the mouse is from a hybrid line (e.g., 50% BALB/c-50% 12954/Sv; or 50% C57BL/6-50% 129). The disclosure includes parental modified non-human mammals and their progeny. In embodiments, the modified mammals are any member of the order Rodentia including but not limited to mice, rats and rabbits. In embodiments, the modified mammals are Mus musculus.

In some embodiments, the animal is a rat. The rat can be selected from a Wistar rat, a Long-Evans Agouti strain, a Sprague Dawley strain, a Fischer strain, F344, F6, and Dark Agouti strain.

In some embodiments, the rat strain is a mix of two or more strains selected from the aforementioned group of rats.

With respect to the present disclosure as compared to prior work, in embodiments, the disclosure includes the proviso that any construct/modified mouse lacks a H1 domain of the murine constant region, and is sufficient to produce single-chain antibodies in rodents that resemble camelid HCAbs or shark IgNar Abs. In an embodiment, the CH1 of Ighg3 is deleted. In an embodiment, the CH1 of Ighg3 is the only deletion. Deletion of CH1 in other constant regions may also be deleted and produce similar HCABs. In embodiments, the disclosure includes the proviso that any construct/modified mouse described herein can be designed such a described polynucleotide is not randomly integrated into a chromosome. In embodiments, the described constructs/modified mouse comprise VHH with a human D and J integrated into an endogenous mouse heavy chain locus that is operably linked to an endogenous mouse constant region with at least a CH1 domain deletion. Thus, in embodiments, a described construct/modified mouse does not require knock-out of Ig loci, unlike a previous approach in mice which involved use of a YAC transgene, such as described in Teng, Y., et al., Diverse human VH antibody fragments with bio-therapeutic properties from the Crescendo Mouse. New biotechnology 55, 65-76 (2020), wherein the modified mouse is referred to as the Crescendo Mouse. Thus, the present disclosure provides in certain embodiments for genetic changes that are made directly in the endogenous murine IgH locus. As a consequence, the disclosure enables replacement of the murine IgH, requiring no further genetic alterations or mouse genetic background considerations. The endogenous location within the IgH locus also enables affinity maturation of the naïve repertoire via somatic hypermutation within the context of germinal center response. In embodiments, the disclosure includes the proviso that the described constructs/modified mouse do not include a knock-in at the mouse IgH locus with a selection of VHH elements replacing endogenous murine VH elements, such as that described in Xu, J., et al. Nanobodies from camelid mice and llamas neutralize SARS-CoV-2 variants. Nature 595, 278-282. In this approach, Cmu and Cg1 constant regions had the CH1 encoding exons targeted to enable light chain independent expression of the heavy chain. This differs from the presently described Model III in the selection of VHHs, lack of humanized DH and JH elements and presence of IgM, whereas in one embodiment the present disclosure ablates IgM and IgD, leaving expression of IgG3(—CH1)). These are significant differences as humanization of the DH and JH dramatically enhances translational applicability of single-domain antibodies generated in presently described modified mice. The presence of IgM (—CH1) in the Xu et al model dramatically alters the Ig expression and selection of the B cells. Further, and without intending to be bound by any particular theory, it is believed the majority of the B cells produced by the Xu et al model are of the IgM isotype, and IgG+ B cells also utilize IgG1 but not the IgG3 constant region as set forth in one embodiment of this disclosure. Further, and again without intending to be bound by any particular theory, it is considered that Xu et al. demonstrates a knock-in mouse capable of producing single-chain Abs (IgM —CH1; IgG1 —CH1), but by removing endogenous murine VH elements. It is therefore considered that the Xu et al. model is incapable of producing endogenous VDJs compatible with B cell selection and antibody secretion, in contrast to the present disclosure. Further, embodiments of this disclosure result in production of a wide repertoire of single-chain antibody producing BCRs that use endogenous VH elements with an IgG3 (—CH1) isotype. In embodiments the disclosure includes the proviso that the disclosure does not include replacement of an entire mouse VH locus which prior approaches have shown, but those approaches preclude use of endogenous VH elements in the production of single-chain Abs.

Non-limiting embodiments of the disclosure are illustrated by Model I and Model III. Model I utilizes targeting of embryonic murine genome to mediate a ˜65kb deletion eliminating exons encoding mouse IgM, IgD, and the CH1 exon of IgG3, which are replaced with a selectable marker cassette, illustrated using a Neo cassette, but other selectable markers can be used, such as puromycin N-acetyltransferase (pac), Blasticidin S deaminase (bsd), Neomycin (G418) resistance gene (neo), Hygromycin resistance gene (hygB), Zeocin resistance gene (Sh bla), the HSV1-TK gene and the HPRT1 gene. The inserted selectable marker is configured such that it is flanked by recombinase recognition sequences. Thus, when a suitable recombinase is present in the nucleus the selectable marker can be excised by the recombinase and the chromosome repaired by the appropriate repair mechanisms within the nucleus. The recombinase may be provided by mating a mouse having a chromosome comprising the Model I construct with another mouse that expresses the recombinase. For instance, if the recombinase recognition sequences Frt sites, the mice may be mated with Flp recombinase expressing mice, e.g., Flp deleter mice. The recombinase recognition sites can be other sites, provided a suitable recombinase is provided to remove the marker cassette. Non-limiting examples of such alternative recombinase systems include Cre recombinase that is used with lox sites, such as loxP and LoxM sites; Dre recombinase which functions in the Dre-rox system; Vika recombinase which functions in the Vika/vox system, or a BxB1 recombinase that functions with attP/attB sites. The recombinase may be inducible. In embodiments, expression of the recombinase may be controlled by a repressor. In embodiments, expression of the recombinase may be from an inducible promoter that is operably linked to the sequence encoding the recombinase. The DNA sequences of a wide variety of inducible promoters for use in eukaryotic cells are known in the art, as are the agents that are capable of inducing expression from the promoters. In embodiments, engineered regulated promoters such as the Tet promoter TRE which is regulated by tetracycline, anhydrotetracycline or doxycline, or the lad-regulated promoter ADHi, which is regulated by IPTG (isopropyl-thio-galactoside) may also be used. In embodiments, the activity or localization of the recombinase can be regulated. These embodiments include but are not limited to the use of tamoxifen-based relocalization of a recombinase to the nucleus or ligand-induced dimerization of the enzyme. In Model I, the Neo selection is inserted using two CRISPR guide RNAs per side and Cas9, but other approaches can be used to insert the described constructs, as described further below. Briefly, for Model III, which is described further below, and in the Examples, a targeting vector is constructed so that upon correct targeting into a mouse embryonic cell genome, almost all of the mouse DH (except Ighd1-1), all of the mouse JH, IgM, IgD, and the CH1 domain of IgG3 are targeted and replaced by a genetically engineered DNA fragment. In a non-limiting embodiment, the targeting vector construct comprises 2.4 kilobases of human IGHV 3-30 promoter, Llama VHH2, and down-stream sequence of human IGHV 3-30, 5 kilobases of human IGHV1-3 promoter, Alpaca VHH2, down-stream sequence of human IGHV 1-3, which includes human endogenous regulatory sequences comprising 13 kilobases of human IGHV1-2 promoter, Llama VHH1, down-stream sequence of human IGHV 1-2, which includes endogenous human regulatory sequences 47 kilobases of human IGHV6-1 promoter, which includes regulatory sequences, Camel VHH1 and down-stream sequence of human IGHV 6-1 the entire human DH locus containing all the IGHD elements, the entire human JH locus containing all the IGHJ elements, a Neo selection marker, which is deleted in ES cells by the expression of flp enzyme in the course of ES cell expansion stage, but other recombinase recognition sites and recombinases can be used, as discussed above. The resulting locus generates HCAbs due to the deletion of CH1 and can utilize VHH 1 and 2 domains from mouse, alpaca, camel, and llama fused to human D and J domains and mouse CH2 and CH3 domains.

In an embodiment, the disclosure comprises deletion of the Sμ, and Sγ switch regions to prevent isotope class switching, thereby facilitating expression of only HCAbs without deleting the CH1 domain in other isotypes.

For Model I, design of the guide RNAs were such the borders of the deletion and replacement with the marker cassette resulted in deletion of mouse IgM, IgD and IgG3 CH1, while leaving CH2 and CH3 intact. Also left intact are Adam6, the IgG3 hinge, and the Emu enhancer. The resulting modified locus generates HCAbs due to the deletion of CH1, mimicking camelid CH1 skipping, which prevents use of the light chain during antibody assembly. Other conventional antibody production is ablated due to the deletion of IgM and IgD. Thus, modified mice of this disclosure produce HCAbs that utilize murine VH elements but do not pair with light chain. Model I includes a genetically modified non-human animal, wherein the genome of the animal comprises: at least one unarranged immunoglobulin heavy chain variable domain, at least one unarranged immunoglobulin heavy chain D domain, and one unarranged immunoglobulin heavy chain J domain that are operably linked to a functional non-human immunoglobulin heavy chain constant gene sequence, wherein the CH1 domain from constant region is deleted, enabling expression of single immunoglobulin heavy chain on a B cell independent of light chain. Model III is described further below and in the Examples.

For Model I and Model III, the disclosure includes incorporating additional VHH coding sequences in the targeting vectors. Thus, the targeting vectors and the chromosomes comprising them in inserted form may encode 1, 2, 3, 4, 5, or more xenobiotic variable regions. Furthermore, the disclosure includes, but is not limited to, production of HCAbs that are a product of recombination between the introduced and endogenous variable regions and HCAbs that are a product of mutations and selection that naturally occurs following introduction of an immunogen into the modified mammals. Thus, in embodiments, a diversity of hCAbs can be created to a variety of immunogens. This arises at least in part from rearrangement of VHHs with DH and JH elements to form VDJ, e.g., the variable portion of the of HCAb. Furthermore, junction is imperfect as N/P nucleotides are added during the rearrangement. In embodiments, the described constructs can comprise amino acids that intervene segments of the heavy chain antibodies, or other encoded proteins. Non-limiting examples of intervening sequences include linkers, such as GS linkers, and self-cleaving peptide sequences. A self-cleaving amino acid sequence is typically about 18-22 amino acids long. Any suitable sequence can be used, non-limiting example of which include T2A, P2A, E2A and F2A, the sequences of which are known in the art. In embodiments, exons of the constant regions are a construct/mouse of this disclosure are murine.

The disclosure includes providing a described modified mammal, introducing an immunogen to stimulate production of the HCAbs, and optionally isolating the produced HCAbs and determining their sequences. This allows recombinant production of the produced HCAbs and their use in any of a wide variety of therapeutic, prophylactic, and diagnostic approaches. In embodiments, variable regions, including the entire variable region or CDR1, CDR2, or CDR3, produced according to the described methods can be recombinantly incorporated into other types of binding agents, non-limiting examples of which include antigen-binding (Fab) fragments, Fab′ fragments, (Fab′)2 fragments, Fd (N-terminal part of the heavy chain) fragments, Fv fragments (two variable domains), dAb fragments, isolated CDR regions, single-chain variable fragment (scFv), and other antibody fragments that retain antigen binding function. In embodiments, one or more of the variable regions of the identified HCAbs are provided as a component of a Bi-specific T-cell engager (BiTE), bispecific killer cell engager (BiKE), or a chimeric antigen receptor (CAR), such as for producing chimeric antigen receptor T cells (e.g., CAR T cells).

In embodiments, HCAbs of this disclosure may be used for antibody-dependent cell mediated cytotoxicity (ADCC) and thus may function to kill targeted cells by cell-mediated responses by any of a variety of effector cells.

In embodiments, HCAbs produced by the described modified non-human animals may be modified to carry drugs or toxins, and thus HCAbs may be provided as immunotoxins, or in the form of antibody-drug conjugates (ADCs).

In embodiments, HCAbs produced by the described modified non-human animals may be modified to comprise a detectable label, which can be used for diagnostic or therapeutic purposes.

In embodiments, HCAbs produced by the described modified non-human animals can be delivered as mRNA or DNA polynucleotides that encode the HCAbs for therapeutic purposes, such as by using viral vectors.

In embodiments, HCAbs produced by the described modified non-human animals may be modified such that at least as segment of an HCAb that can bind to an antigen with specificity is present in a fusion protein. A component of the fusion protein may provide for improved therapeutic uses, such as by extending half-life, bioavailability, or safety.

In embodiments, the methods of this disclosure are performed using the described DNA constructs and involve the participation of certain proteins to insert the described targeting vectors, which may be provided as circularized or linear DNA molecules. In embodiments, the protein may be produced within the cell via expression of any suitable expression system that encodes the protein. In embodiments, any protein required to participate in the described process may be modified such that it includes a nuclear localization signal. In embodiments, a protein may be administered directly to the cells. For proteins that require an RNA component to function, such as certain Cas proteins as described below, the protein(s) and the RNA component may be administered to the cells as ribonucleoproteins (RNPs). In embodiments, the targeting vector is introduced into the described locus using any designer nuclease. In embodiments, the nuclease is a RNA-guided CRISPR nuclease. A variety of suitable CRISPR nucleases (e.g., Cas nucleases) are known in the art, as are methods for designing and selecting appropriate guide RNA constructs so that homology arms can be precisely inserted at a predetermined location using a Cas nuclease. Thus, in embodiments, an RNA-guided Cas nuclease may be used. In an embodiment, two guide RNAs may be included so that the locus is modified in two positions.

In embodiments that involved use of a CRISPR mediated modification, the Cas is selected from a Class 1 or Class 2 Cas enzyme. In embodiments, a Type I, II, III or CRISPR Cas nuclease is used. In specific and non-limiting embodiments, the Cas comprises a Cas9, such as Streptococcus pyogenes (SpCas9). Derivatives of Cas9 are known in the art and may also be used to introduce the LP into a locus. Such derivatives may be, for example, smaller enzymes that Cas9, and/or have different proto adjacent motif (PAM) requirements. In a non-limiting embodiments, the Cas enzyme may be Cas12a, also known as Cpf1, or SpCas9-HF1, or HypaCas9.

In embodiments, such as in Model III, the methods can be performed without providing exogenous nucleases such that the described constructs are inserted into a selected locus by homologous recombination in the absence of introduced nucleases. Homologous recombination proceeds using first and second homology arms. For CRISPR implemented methods, the first and second homology arms can include sequences that are recognized and cleaved by the same Cas-mediated cleavage system that recognizes and cleaves the chromosomes. Cas cleavage sites may be positioned at or near the end of the homology arms.

With respect to the homology arms, their length is not particularly limited, provided they have a length that is adequate for homologous recombination to occur when nuclease-mediated cleavage of the selected locus occurs. In embodiments, the homology arms have a length of from 100 bp-1 Kbp, inclusive, and including all integers and ranges of integers there between.

For certain embodiments, a targeting vector may also be inserted into the described locus using non-Cas based nuclease approaches. Suitable examples include but are not necessarily limited to zinc-finger nucleases and MADzymes. Non-limiting examples of MADzymes known in the art include MAD2 and MAD7 and are included in the Cas12a category of nucleases.

In embodiments, cells modified according to this disclosure comprise a heterozygous, or homozygous insertion of the described targeting vectors. In embodiments, homozygous insertions are obtained by mating heterozygous mammals and selecting homozygous progeny.

### EXAMPLES

The disclosure is further described in the following examples, which do not limit the scope of the invention. Conventional methods that known in the art are not described in detailed in the Examples such as PCR, molecular cloning techniques, bacteria BAC engineering, embryonic stem cell culture, micro-injection, animal breeding, and the like. CDR3s were identified. Examples 1-6 refer to Model III. Example 7 refers to Model I.

### Example 1

### Generation of Camelid-Human Immunoglobulin Heavy Chain Mice

In this example, a mouse was modified so that the mouse contained a nucleic acid sequence comprising VHHs from camel, alpaca, and llama and entire human Ds and Js operably linked to a mouse Immunoglobulin C region.

Specifically, the mouse was modified so that the non-human animal contained a nucleic acid sequence comprising VHHs from camel, alpaca, and llama and entire human Ds and Js operably linked to a mouse Immunoglobulin C region in the endogenous locus.

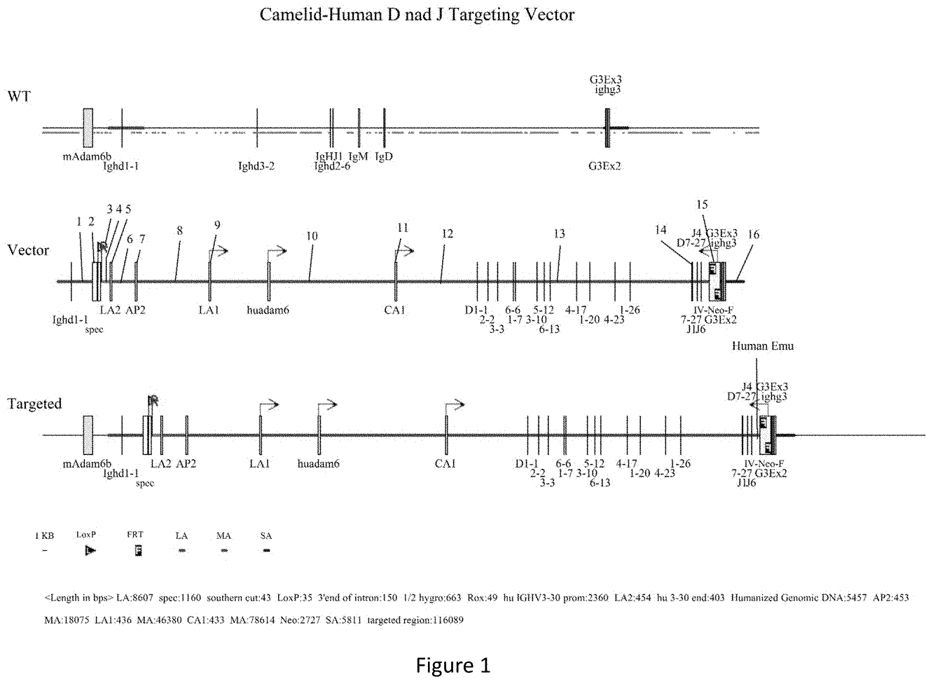

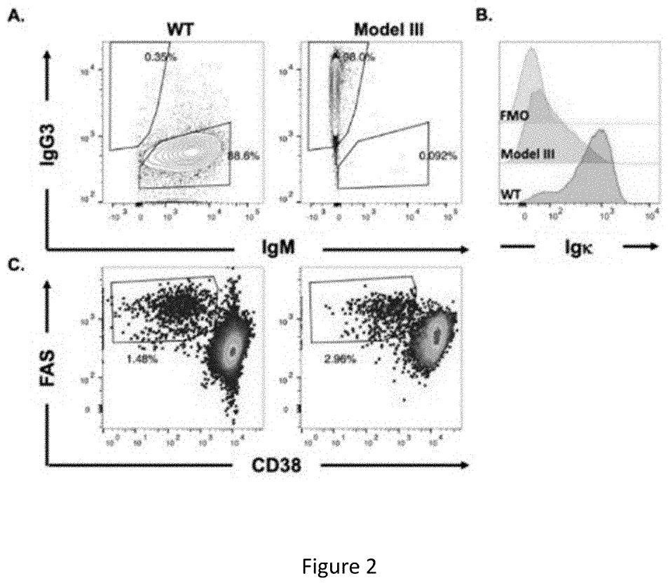

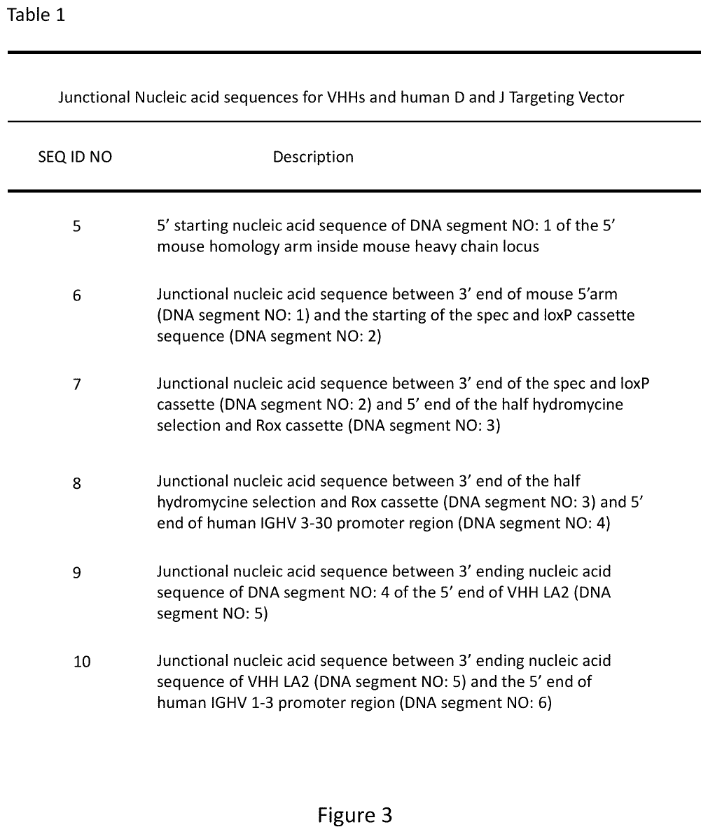

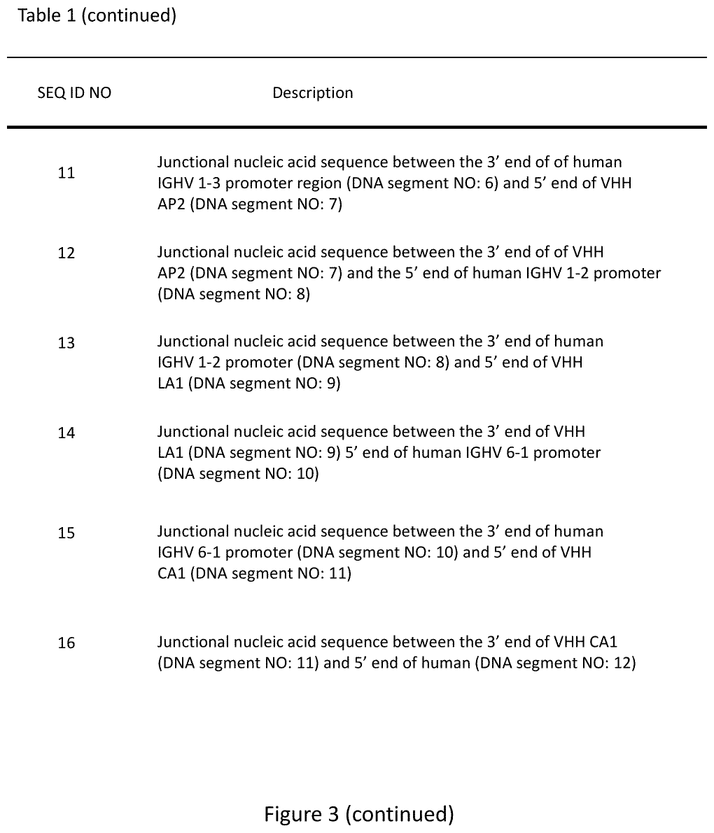

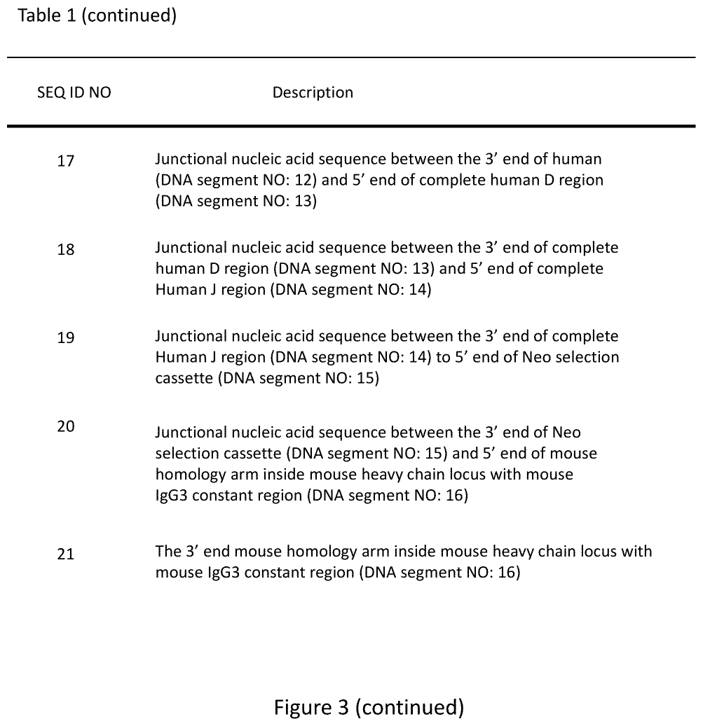

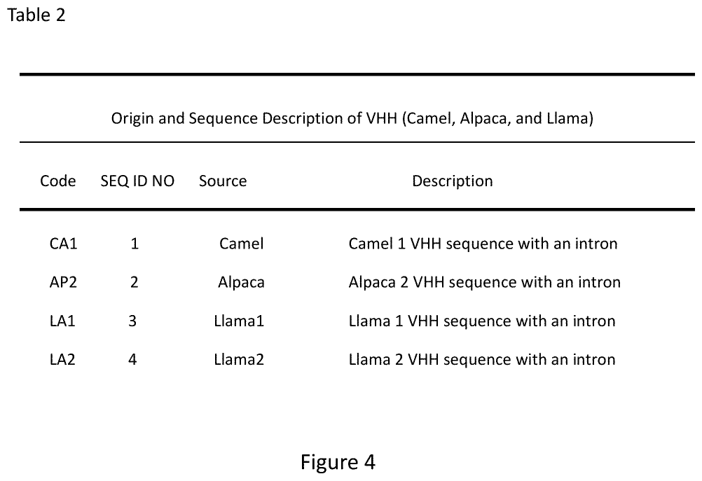

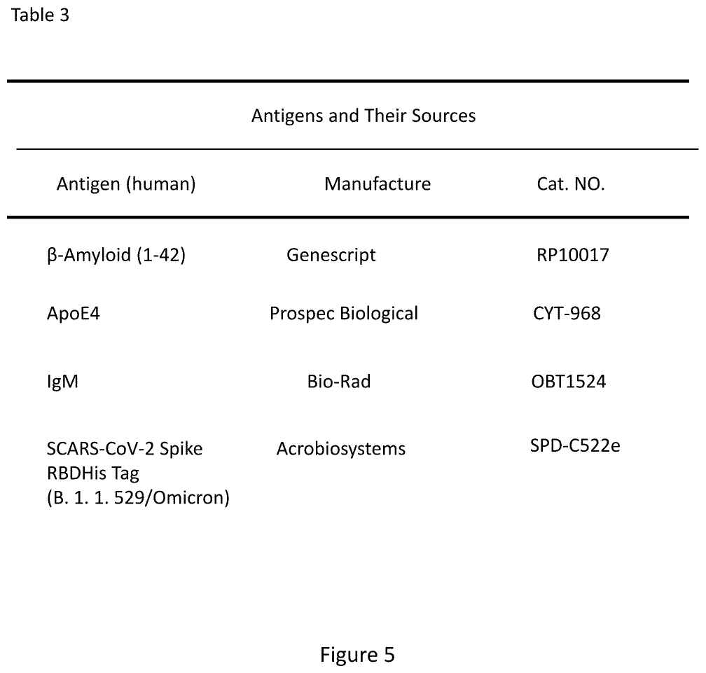

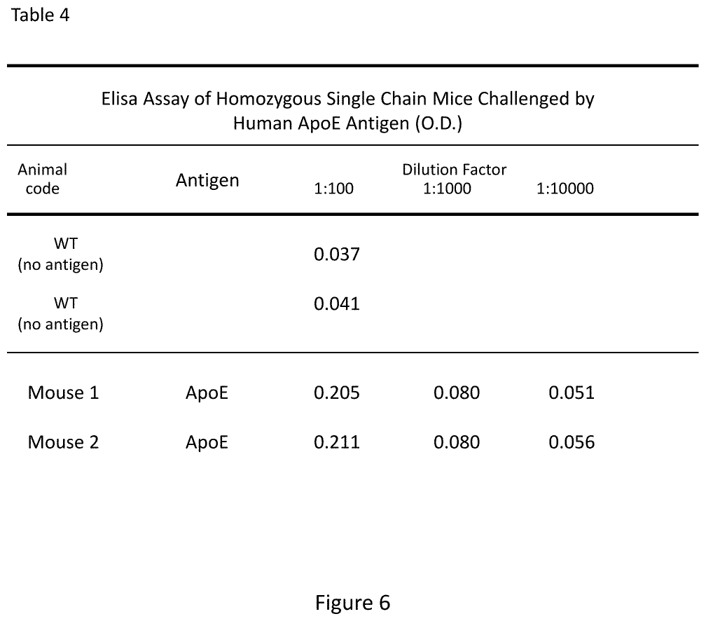

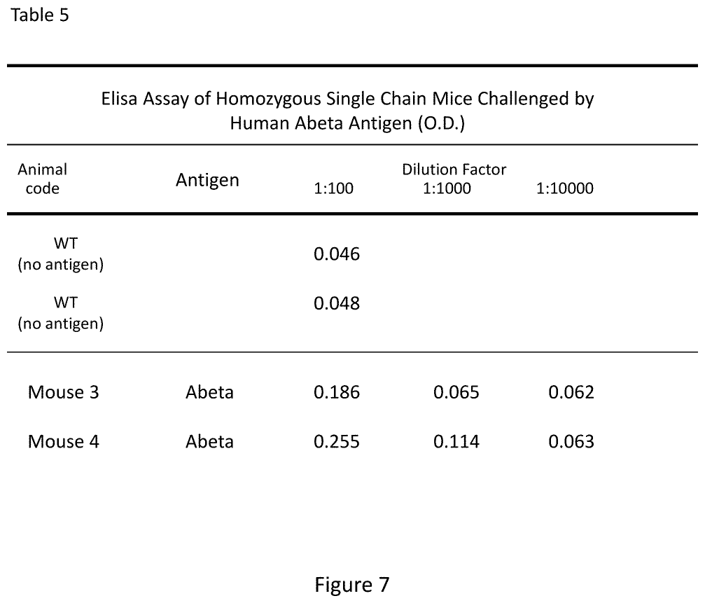

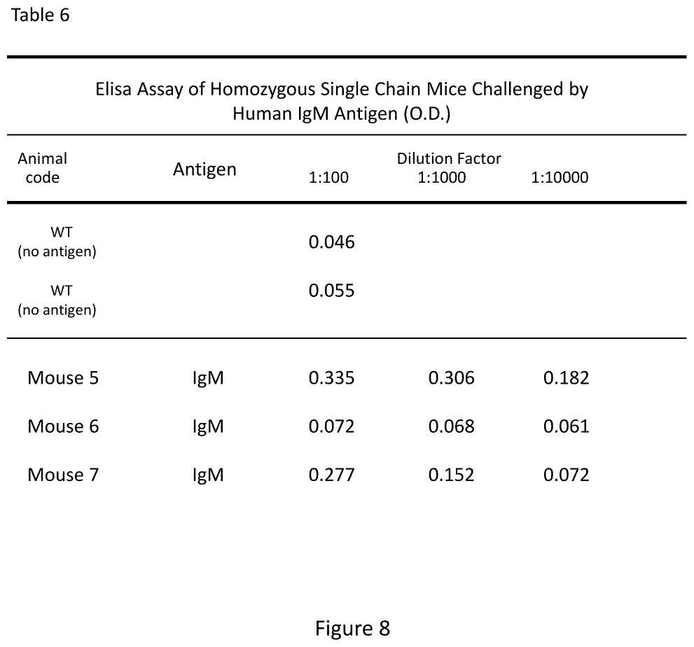

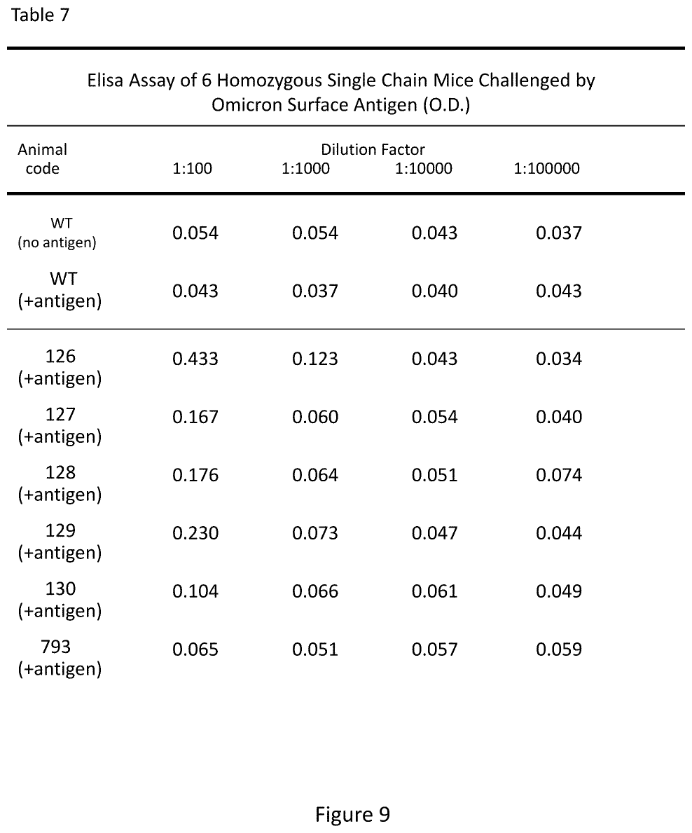

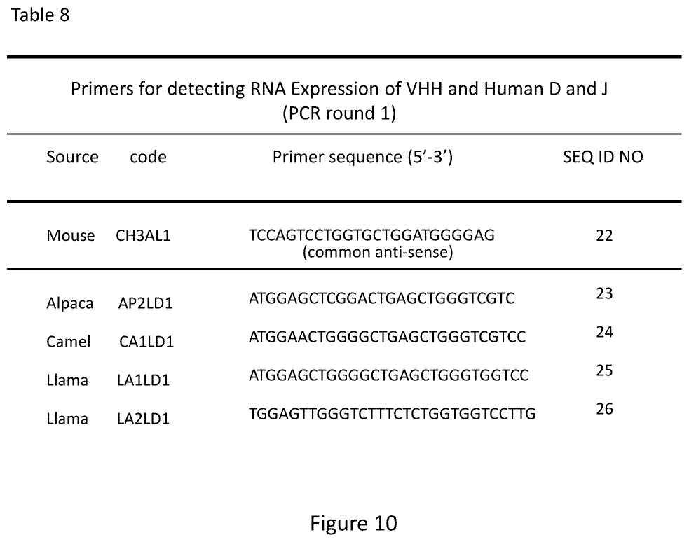

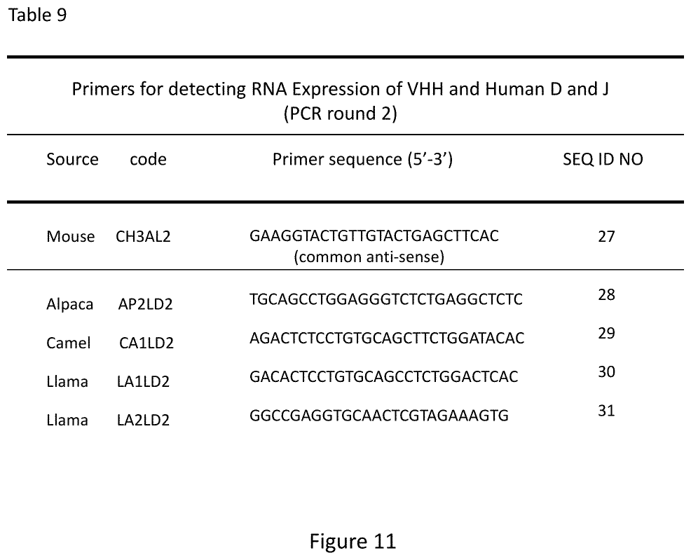

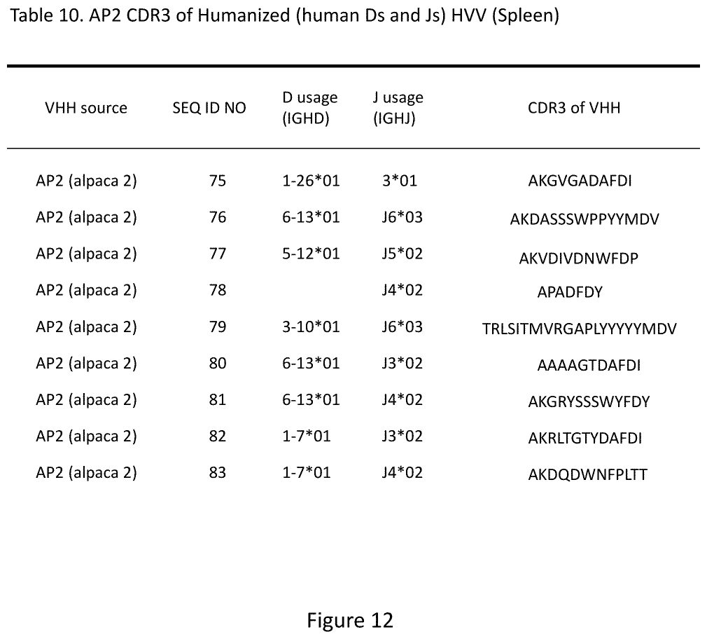

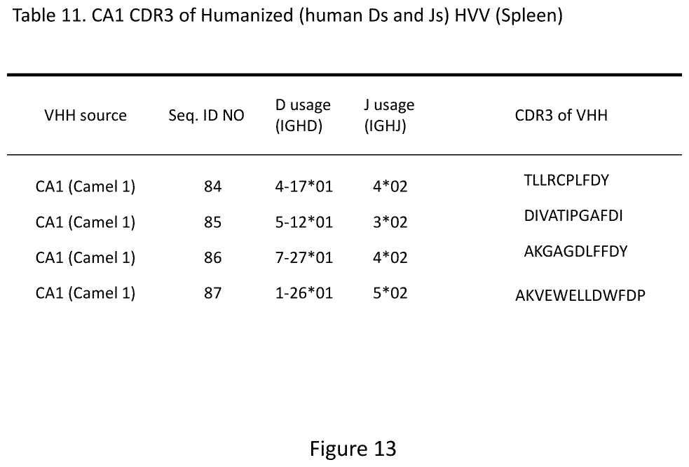

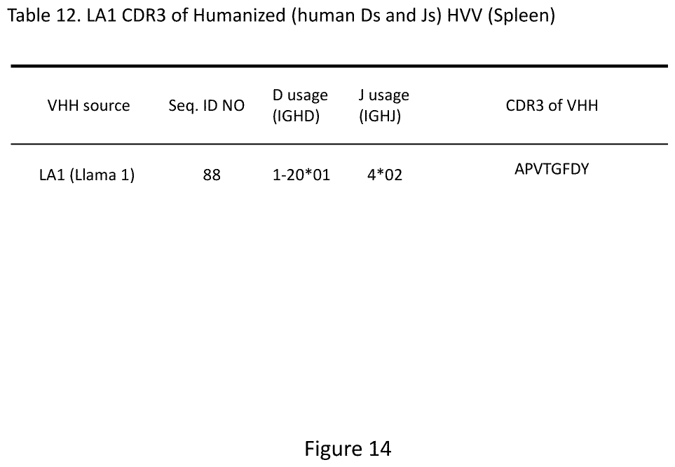

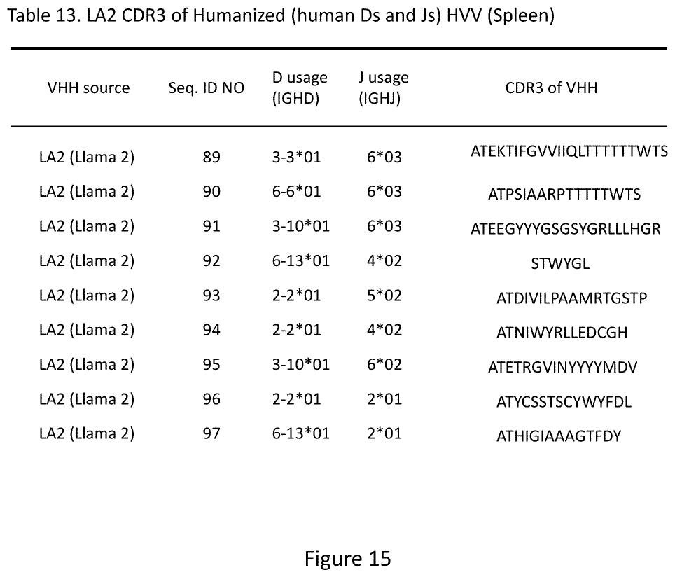

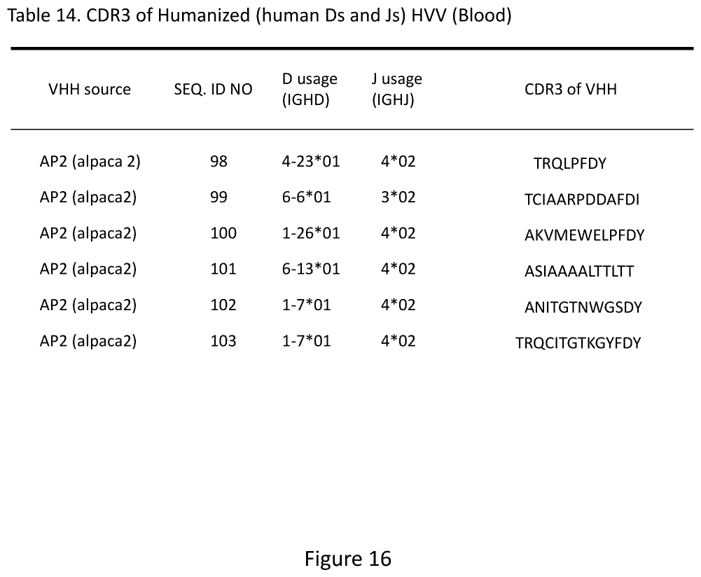

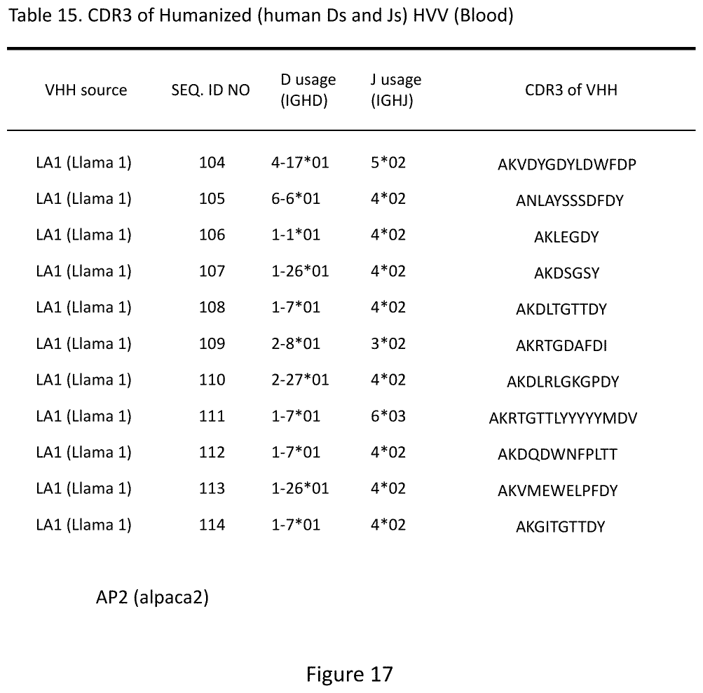

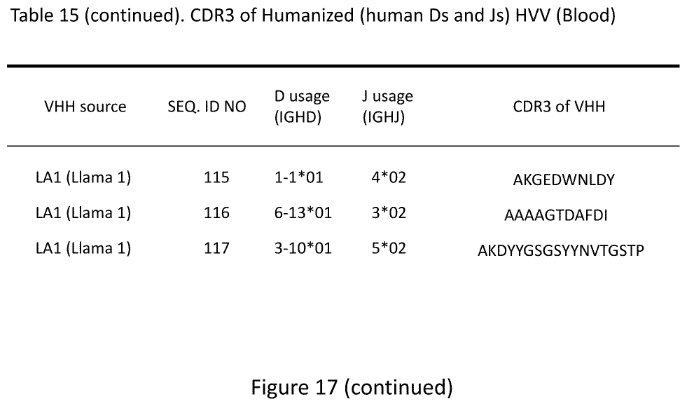

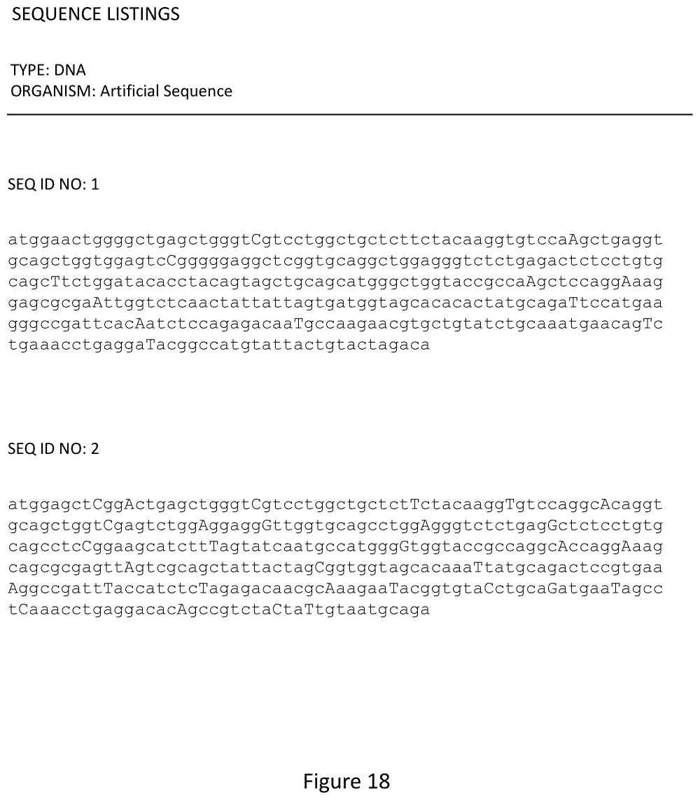

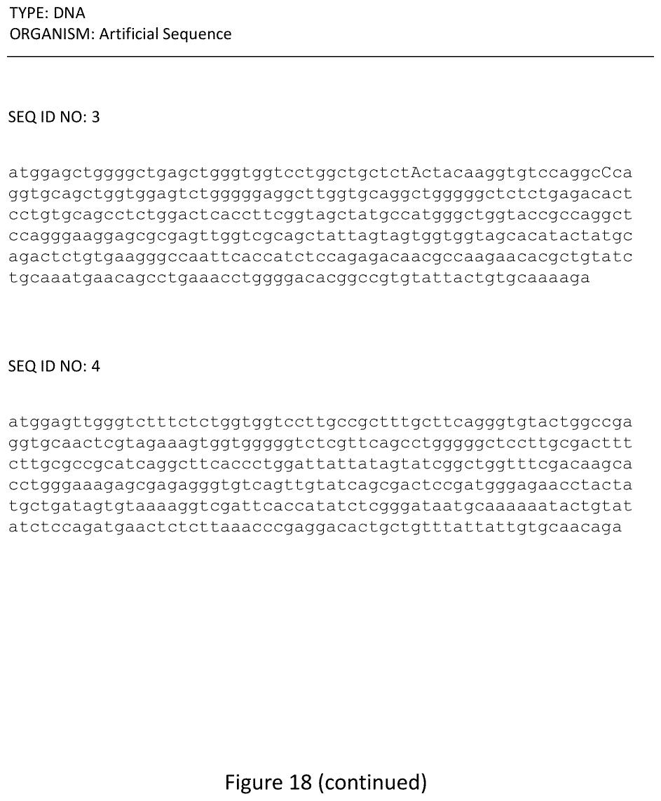

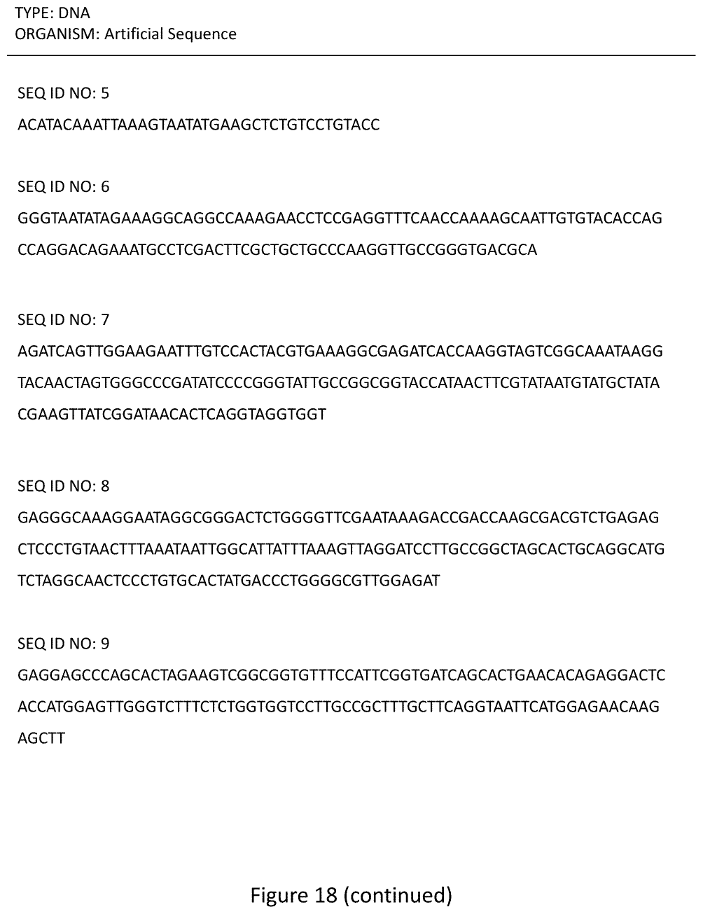

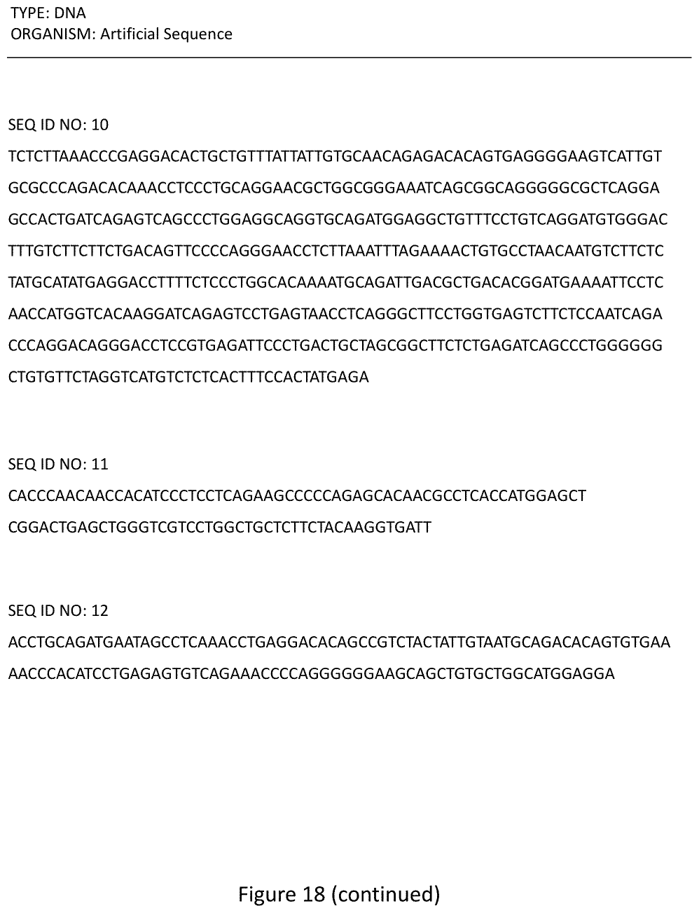

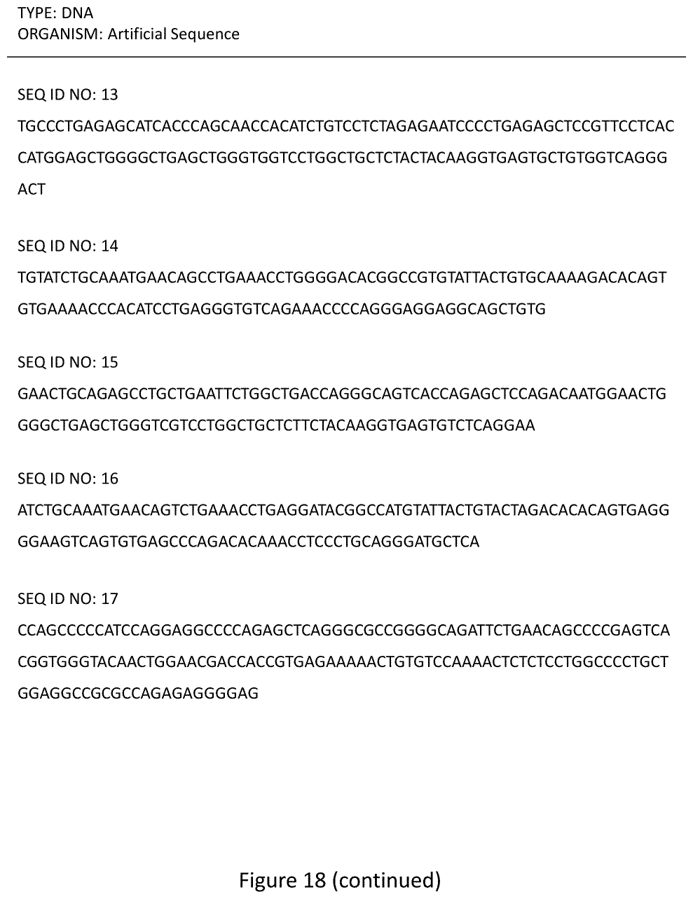

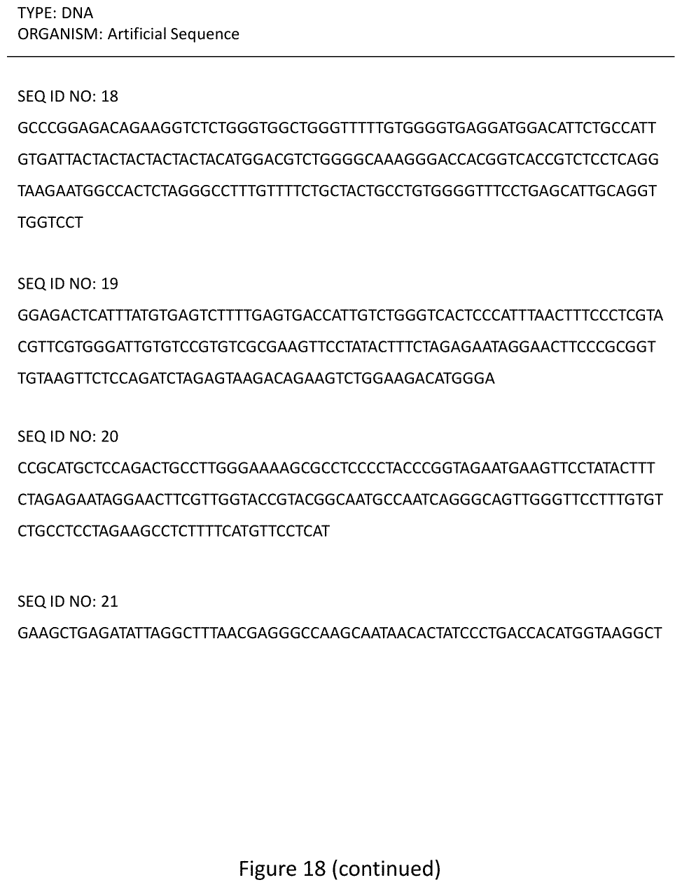

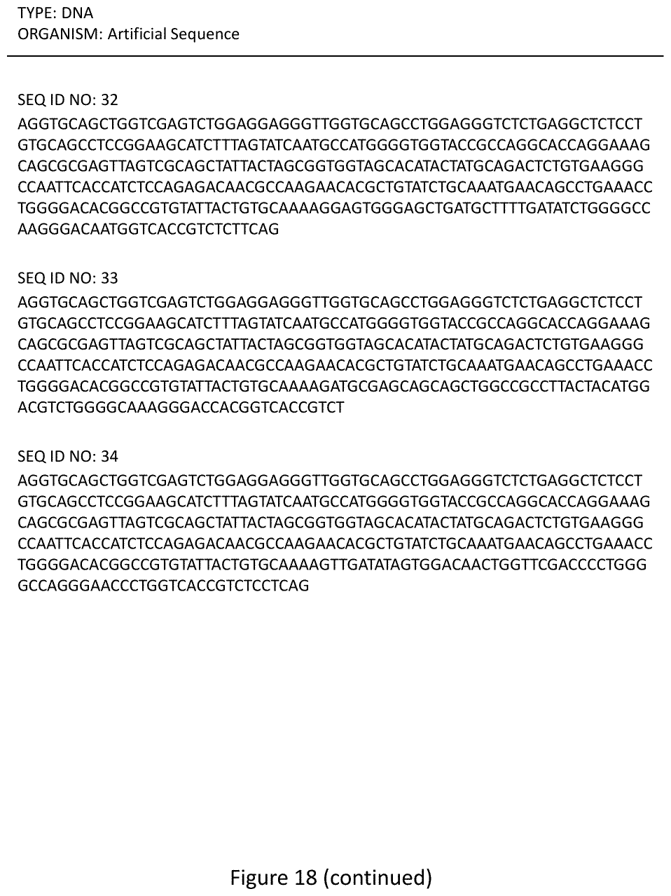

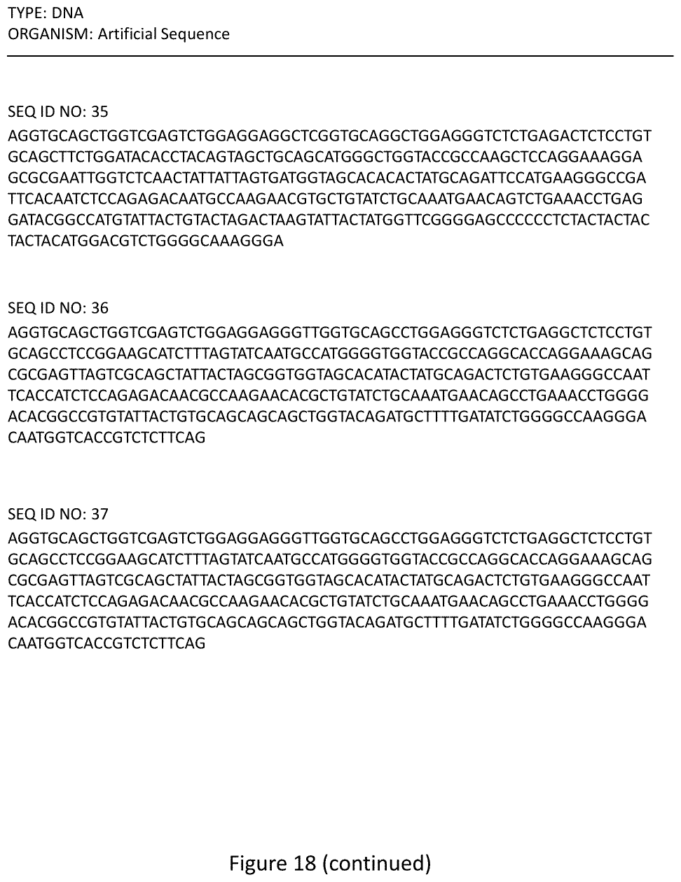

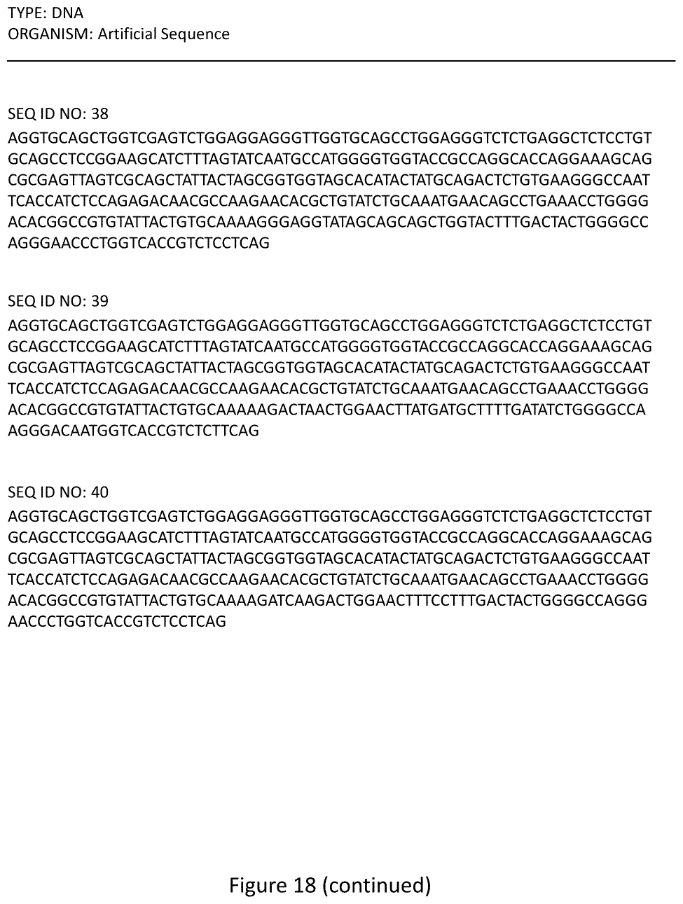

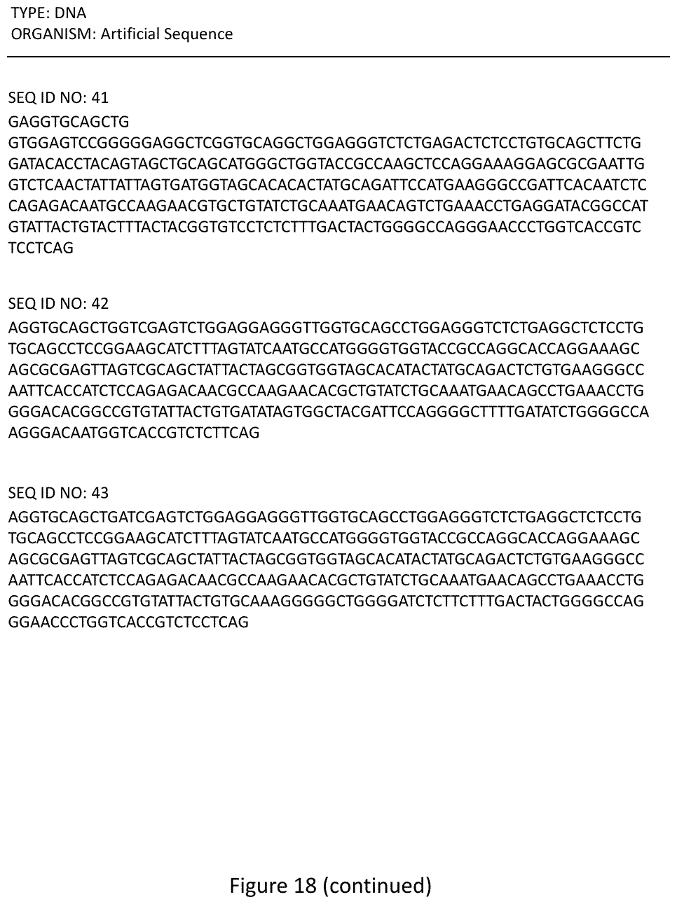

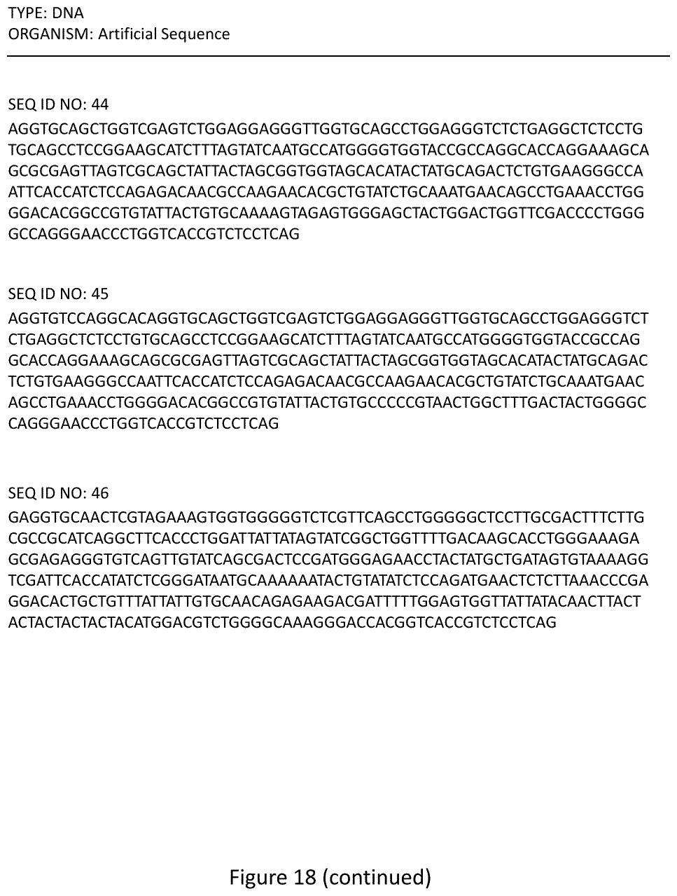

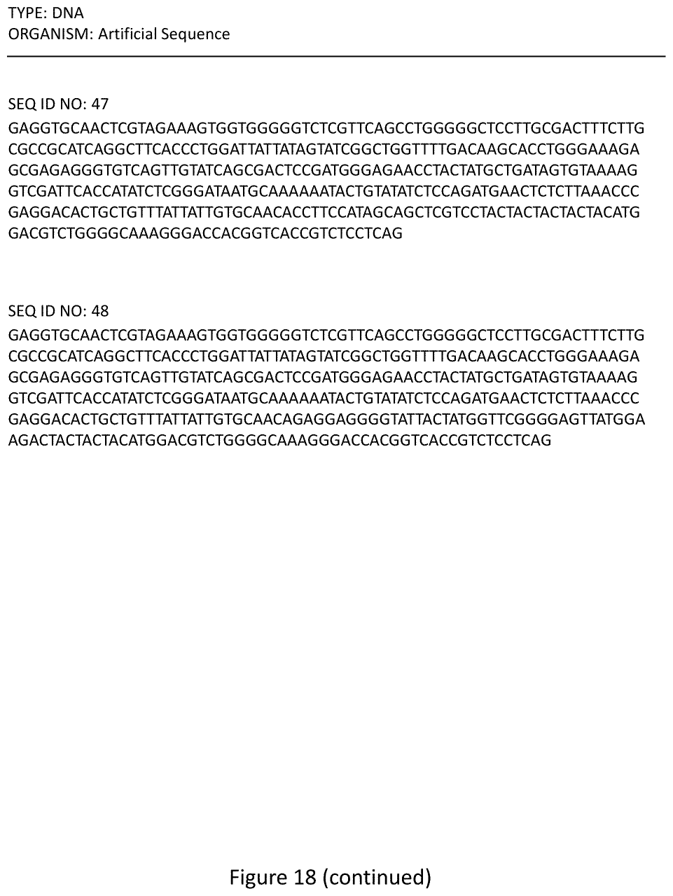

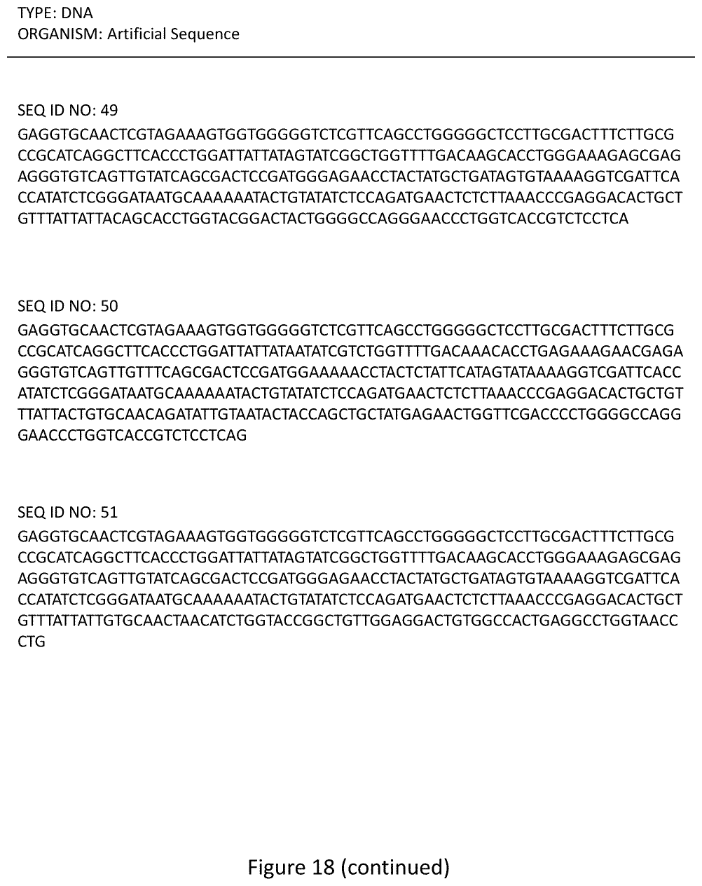

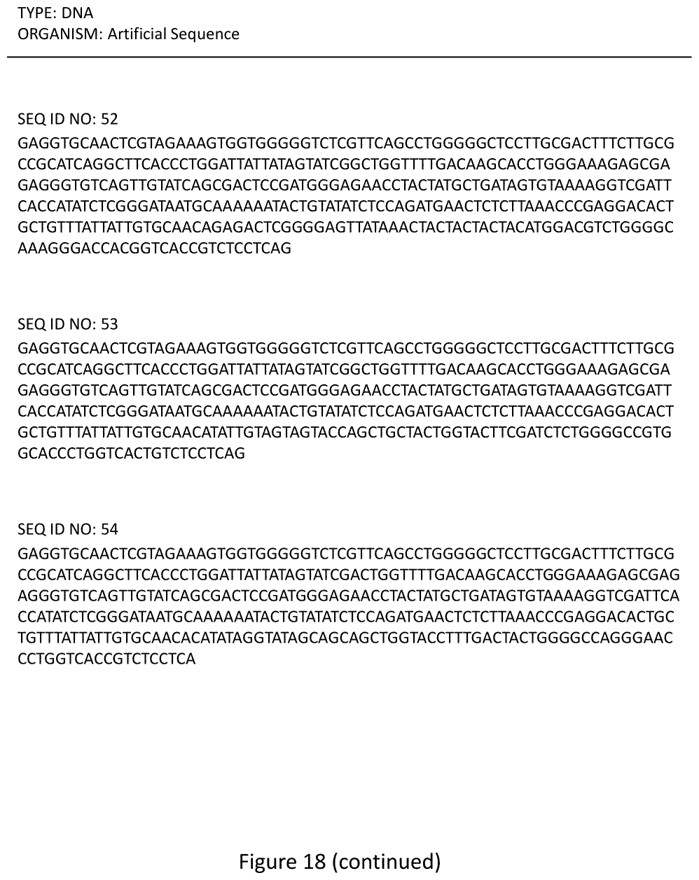

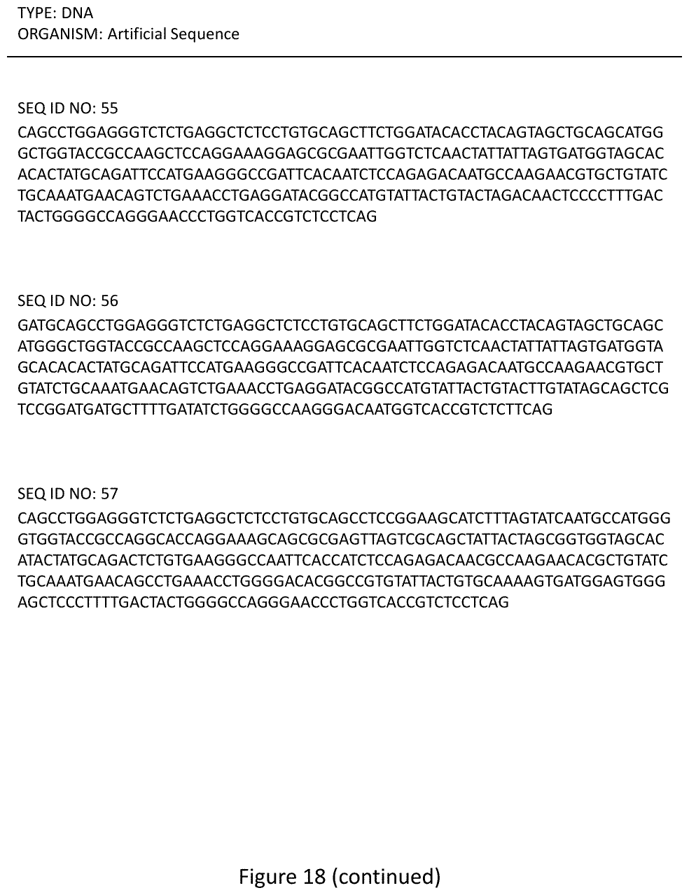

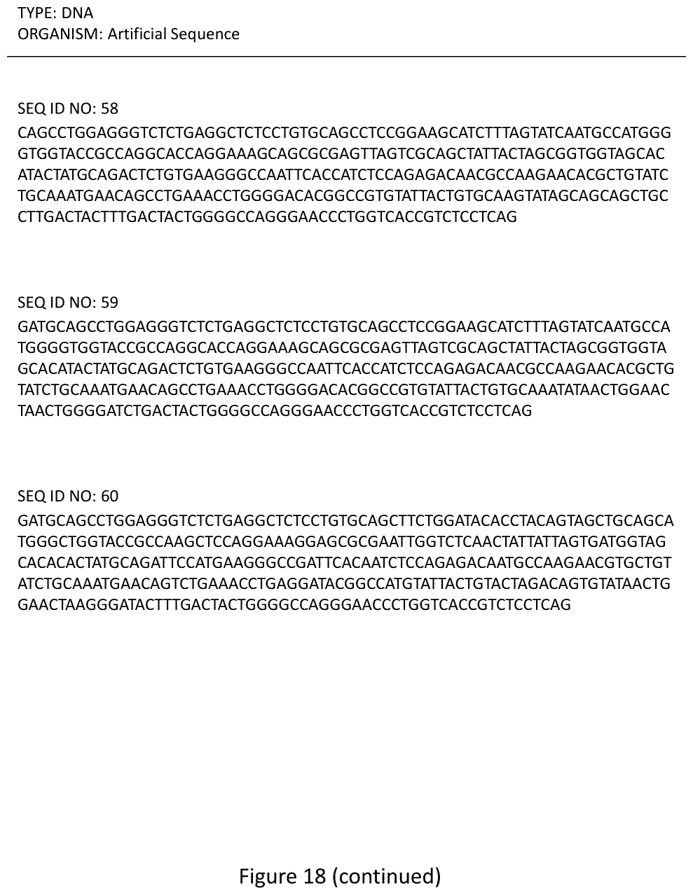

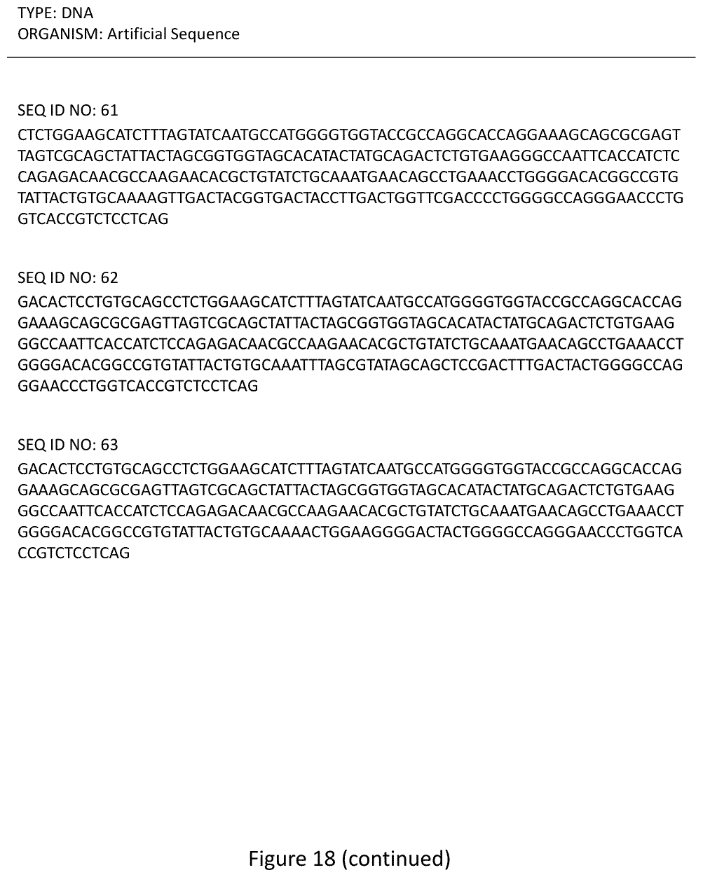

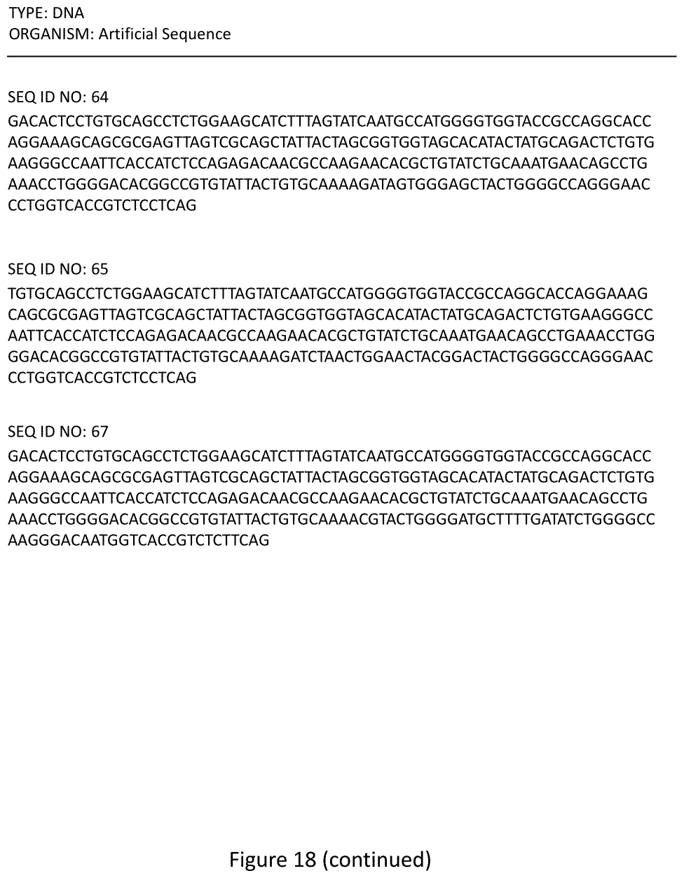

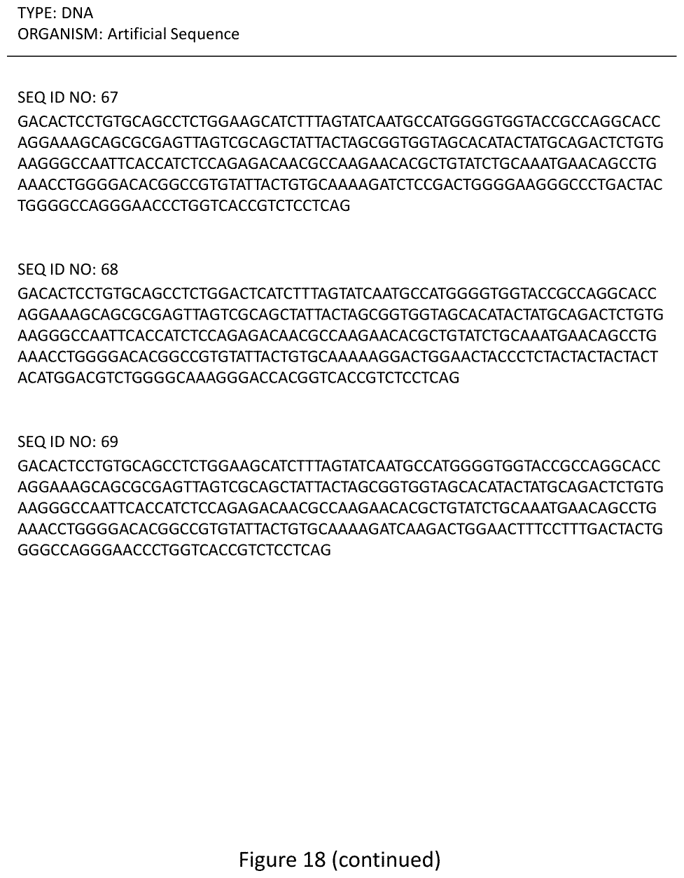

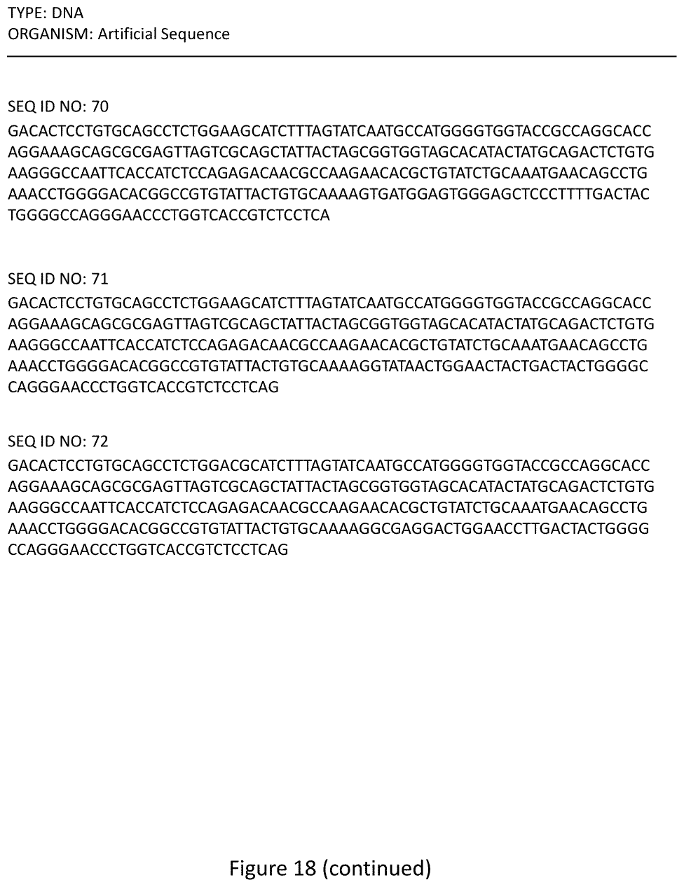

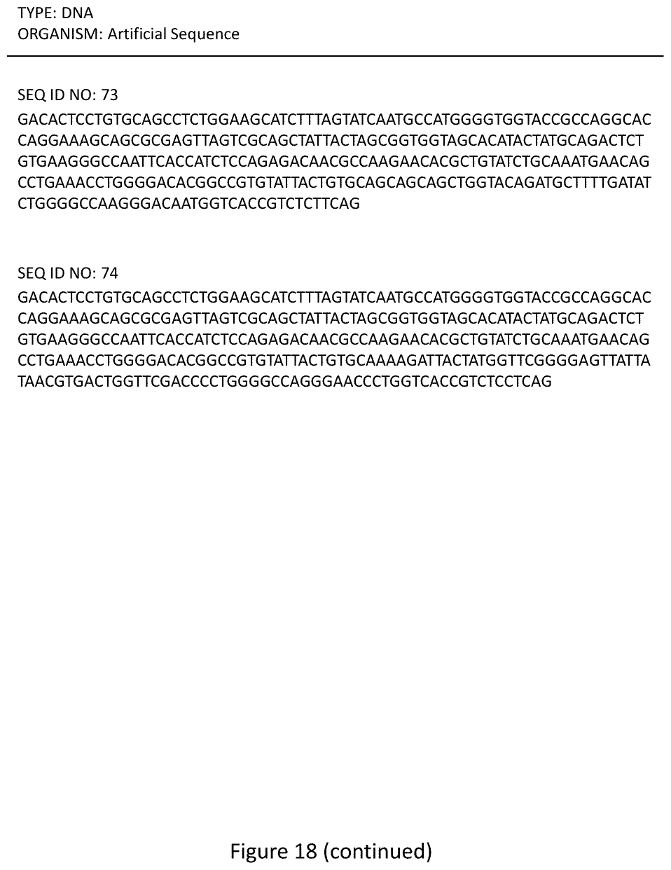

More specifically, 116 kb of mouse sequence including most mouse Ds, entire mouse Js, mouse IgM, mouse IgD, and mouse CH1 was replaced with 157 kb of chimeric sequences corresponding to 4 camelid VHH invariable domains and their human promoters, entire human Ds, and entire human Js (FIG. 1). Because the human sequence stops just after last human J, the human IgM, IgD, IgG3 switch region and human CH1 domain were not included in this animal. The replacement event also removed mouse CH1 domain, so there is no CH1 domain from either human or mouse immunoglobulin heavy chain in this animal. Therefore, after the replacement event VHHs from camel, alpaca, and llama and entire human Ds and Js operably linked to a mouse Immunoglobulin C region (e.g., IgG3).

The camelid-humanization strategy was summarized in FIG. 1. Junctional nucleic acid sequences inside targeting vector among camelid, mouse, human, and prokaryotic/eukaryotic selection cassette are summarized in Table 1 and also provided in the Sequence Listing (SEQ ID NO: 5-21).

Specifically, the first step of generation of a genetically modified mouse with camelid-humanization was to construct a camelid-humanization heavy chain targeting vector (Vector ID NO: 1). The targeting vector (Vector ID NO: 1) has 16 DNA segments: DNA seg. 1: A 5′ mouse homology arm. DNA seg. 2: A prokaryotic selection cassette with a loxP recombinant site, which can serve as a further deletion or additional DNA insertion landing pad. DNA seg. 3: A half hygromycine selection cassette and a Rox recombinant site to facilitate future DNA landing. DNA seg. 4: A human IGHV 3-30 promoter DNA sequence. DNA seg. 5: A VHH from Llama 2 (DNA SEQ ID NO: 4). DNA seg. 6: A human IGHV 1-3 promoter DNA sequence. DNA seg. 7: A VHH from alpaca 2 (DNA SEQ ID NO: 2). DNA seg. 8: A human IGHV 1-2 promoter DNA sequence. DNA seg. 9: A VHH from llama 1 (DNA SEQ ID NO: 3). DNA seg. 10: A human IGHV 6-1 promoter DNA sequence. DNA seg. 11: A VHH from camel 1 (DNA SEQ ID NO: 1). DNA seg. 12: A human DNA sequence connecting the 3′ end of human IGHV 6-1 to the first human heavy chain D (D1-1). DNA seg. 13: A human DNA sequence comprising complete human heavy chain Ds. DNA seg. 14: A human DNA sequence comprising complete human heavy chain Js. DNA seg. 15: A pro/eukaryotic G418/Neo selection cassette. DNA seg. 16: A 3′ mouse homology arm including mouse constant region C4 (from exon 1 to exon 4). Table 2 summarized the origin of VHH and their SEQ ID NO (1-4).

One human BAC clone and two mouse fosmid clones were used for construction of the camelid-humanization heavy chain targeting vector (Vector ID NO: 1) (HumanBAC: CH17-236114 and mouse WI1-921O3 and WI1-1006115). The 16 DNA segments (DNA segment NO: 1-16) was on a BAC based vector and were put together (Vector ID NO: 1) through DNA ligation and bacteria homology recombination using homology arm with average length of 130 bp (various from 50-200 bp). The bacteria homology recombination was helped by using bacterial Spec and Kan selection cassettes. During the targeting vector construction process, an unique (not cutting into DNA segment NO: 2, 6, 7, 4) enzyme cutting site AscI was introduced to allow linearization of the targeting vector (Vector ID NO: 1). The linearized targeting vector (Vector ID NO: 1) was electroporated into a hybrid mouse embryonic stem (ES) cell line derived from a hybrid strain by crossing mice of two different inbred strains (B6 and 129). ES cell with correct targeted clones were identified and confirmed by combination of various methods (e.g., PCR based assays, long arm PCR, long range PCR, southern, and/or human replacement PCR) known in the art. Targeted ES clones were expanded and micro-injected into mouse blastocysts to generate chimera mouse with human targeted gene segment (DNA segment NO: 6). Germline mouse was obtained by further mating the chimera with B6 inbreed and additional PCR confirmation was performed. The Neo cassette was removed by the activity of Flp recombinase ES cells or mouse tissues.

Human Emu was also included inside the targeting vector. It located between DNA segment 14 and 15 as indicated in FIG. 1 on the right side of bottom line (targeted allele).

### Example 2

### Generation of Homozygous for Camelid-Humanization Heavy Chain Mice

Camelid-humanization heavy chain mice achieved in Example 1 were inter-bred. The resulting progeny included WT, homozygous, heterozygous camelid-humanization heavy chain mice. Disappearing of WT PCR band at the replaced region of mouse heavy chain loci was the indication of homozygosity of human transgene.

### Example 3

### ELISA Analysis of Antigen Immunized Camelid-Humanization Heavy Chain Mice

Homozygous camelid-humanization heavy chain mice were immunized by giving biweekly injections of a dose of 100 μg in adjuvant (Freund's complete and incomplete). Complete Freund's adjuvant is only used with the first immunization. Subsequent immunizations are performed in phosphate-buffered saline (PBS) or normal saline, with or without Incomplete Freund's adjuvant. The choice of adjuvant is dependent on the subclass of immunoglobulin required. Over the course of the 6-wk immunization schedule, each animal usually receives a total of six injections (three subcutaneous and three intraperitoneal). Once a good titer has developed against the antigen of interest, regular boosts and bleeds are performed to collect the maximum amount of serum. Boosts were spaced every 2-3 wk, and serum samples of 200-300 μL were collected 10-12 d after each boost. Table 3 shows the antigens and their sources been used for immunization of homozygous camelid-humanization heavy chain mice. A total of 4 antigens cover different areas for research: β-Amyloid (1-42) (e.g., Neuronal research), ApoE (e.g., Neuronal, heart, inflammatory research), IgE or IgM (e.g., Immune research), SCARS-CoV-2 Spike (e.g., Covid and infection disease research).

Table 4 shows the results of ELISA analysis from homozygous camelid-humanization heavy chain mice immunized by human ApoE in mouse number 1 and 2. The single-chain antibody could specifically bind to human ApoE even at a dilution factor of 1:1000 in both mice.

Table 5 shows the results of ELISA analysis from homozygous camelid-humanization heavy chain mice immunized by human Abeta (1-42) in mouse number 3 and 4. Even though short peptide of this antigen (only 42 amino acids), the single-chain antibody could still specifically bind to human Abeta (1-42) at a dilution factor of 1:1000 in at least mouse number 4.

Table 6 shows the results of ELISA analysis from homozygous camelid-humanization heavy chain mice immunized by human IgM in mouse number 5-7. The single-chain antibody could clearly specifically bind to human IgM at a dilution of 1:1000 in all the mice and at least two mice (number 5 and 7) even at a dilution factor of 1:10000.

Table 7 shows the results of ELISA analysis from 6 homozygous camelid-humanization heavy chain mice immunized by SCARS-CoV-2 Spike protein in mouse number 126-130, 793. The single-chain antibody could clearly specifically bind to SCARS-CoV-2 Spike protein at a dilution of 1:1000 in 5 out of 6 mice (mouse number 126-130).

### Example 4

### Analysis of Re-arranged Camelid VHHs, Human Ds, and Js in Camelid-Humanization Heavy Chain Mice

Spleen and blood were collected from homozygous camelid-humanization heavy chain Mice. Cells were first treated with Red Blood Cell Lysis buffer (0.8 g NH4C1 and 0.11 g NaHCO3 in 100 ml water-based solution) to remove red blood cells and white blood cells including B cells were collected by centrifugation. Total RNA was isolated by using RNeasy Mini Kit (Qiagen) according to manufacture suggestions. Total RNA was translated into cDNA by using random priming oligos provide with the Transcriptor First Strand cDNA Synthesis Kit (Roche) in total volume of 15 ul. One microliter of resulting cDNA was used as a template to conduct a first round (round 1) of PCR reaction (total 40 uL) with primer pairs covering camelid VHH region to mouse C region including CDR3 region. For each VHH CDR3 detection, one primer was located at the 5′ end of VHH and second primer was designed and located inside the mouse C region also served as a common anti-sense primer for all the camelid VHH PCR reaction (SEQ ID NO: 22) (Table 8). For example, as shown in table 8, first round PCR of CDR3 of alpaca 2 and human D and J was amplified between a primer (SEQ ID NO: 23) and the common C region primer (SEQ ID NO: 22). First round PCR of CDR3 of camel and human D and J was amplified between a primer (SEQ ID NO: 24) and the common C region primer (SEQ ID NO: 22). First round PCR of CDR3 of llama 1 and human D and J was amplified between a primer (SEQ ID NO: 25) and the common C region primer (SEQ ID NO: 22). First round PCR of CDR3 of llama 2 and human D and J was amplified between a primer (SEQ ID NO: 26) and the common C region primer (SEQ ID NO: 22). Because of the un-purify of the total RNA, a second round PCR is generally needed.

For the second round PCR, two primers were designed within the previous primers described in first round PCR. Typically, a primer 50-100 bp down-stream of the camelid VHH first round primer and a second common primer (SEQ ID NO: 27) (Table 9), which is 50-100 bp up-stream of the common primer of first round (SEQ ID NO: 22). One microliter PCR product from round 1 was introduced as template for second round PCR in a total volume of 40 uL. For example, as shown in table 9, second round PCR of CDR3 of camelid was amplified between a primer (SEQ ID NO: 28) and the common C region primer (SEQ ID NO: 27). Second round PCR of CDR3 of of alpaca 2 and human D and J was amplified between a primer (SEQ ID NO: 28) and the common C region primer (SEQ ID NO: 27). Second round PCR of CDR3 of camel and human D and J was amplified between a primer (SEQ ID NO: 29) and the common C region primer (SEQ ID NO: 27). Second round PCR of CDR3 of llama 1 and human D and J was amplified between a primer (SEQ ID NO: 30) and the common C region primer (SEQ ID NO: 27). Second round PCR of CDR3 of llama 2 and human D and J was amplified between a primer (SEQ ID NO: 31) and the common C region primer (SEQ ID NO: 27).

### Example 5

### Repertoire Analysis of Re-arranged Camelid VHHs, Human Ds, and Js in Camelid-Humanization Heavy Chain Mice

The BCR CDR3 repertoire diversity was confirmed by PCR (Example 4). PCR products were cloned into a plasmid vector. Twelve plasmids from each VHH (Alpaca 2, camel 1, llama 1, and llama 2) were Sanger sequenced. Table 10 shows AP2-Human D and J based CDR3 of Humanized (human Ds and Js) HVV in the spleen. Table 11 shows CA1-Human D and J based CDR3 of Humanized (human Ds and Js) HVV in the spleen. Table 12 shows LA1-Human D and J based CDR3 of Humanized (human Ds and Js) HVV in the spleen. Table 13 shows LA2-Human D and J based CDR3 of Humanized (human Ds and Js) HVV in the spleen. The results from tables 10-13 showed that different camelid HVVs can be fused with different human Ds and different human Js to create high diversified single-chain antibodies in the spleen of the VHH-Human D and J mice.

Camelid VHHs, human Ds, and Js in camelid-humanization heavy chain receptors also found in the B cells of mouse blood. Table 14 shows AP2-Human D and J based CDR3 of Humanized (human Ds and Js) HVV in the blood. Table 15 shows LA2-Human D and J based CDR3 of Humanized (human Ds and Js) HVV in the blood. The results from tables 14 and 15 showed that different camelid HVVs can be fused with different human Ds and different human Js to create high diversified single-chain antibodies in the blood of the VHH-Human D and J mice.

### Example 6

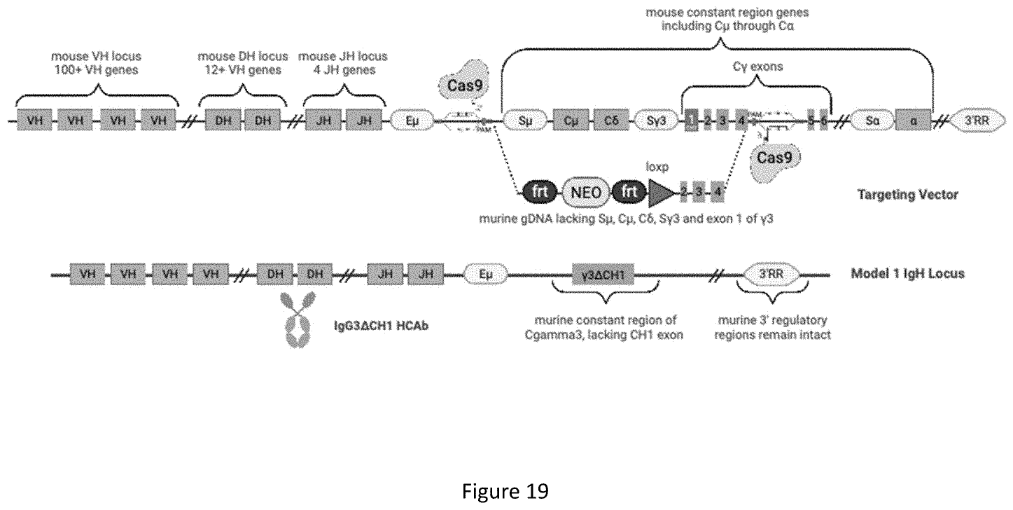

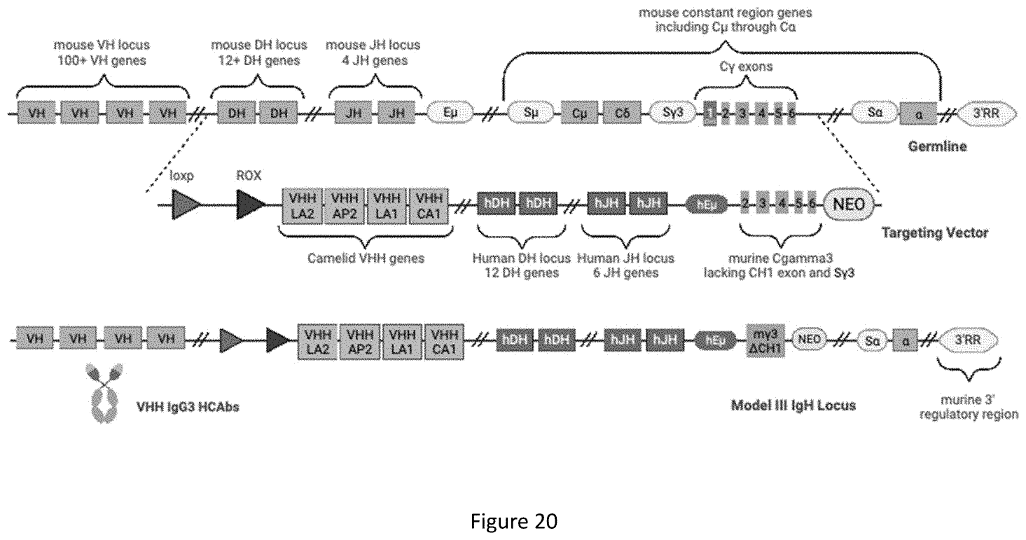

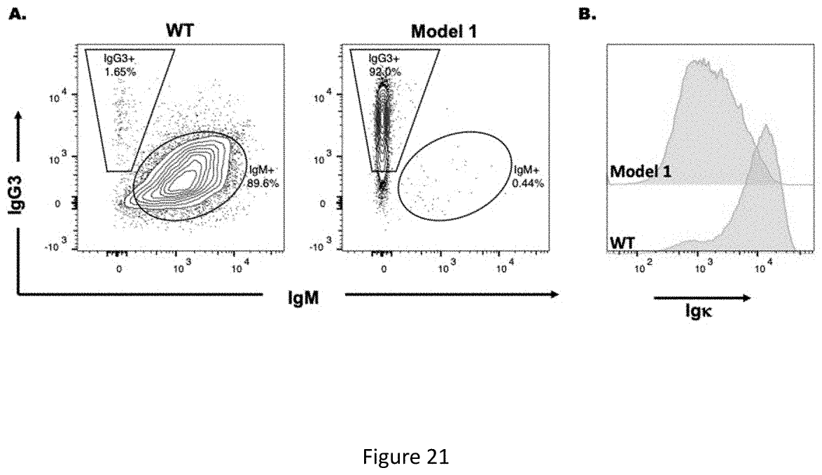

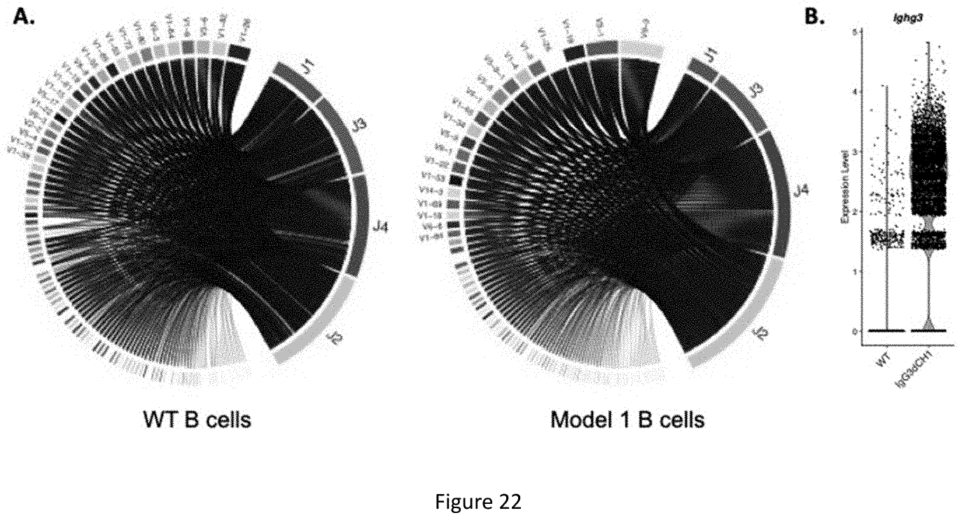

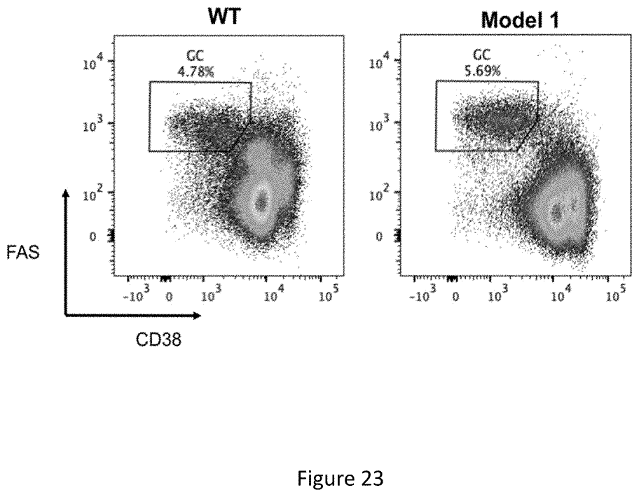

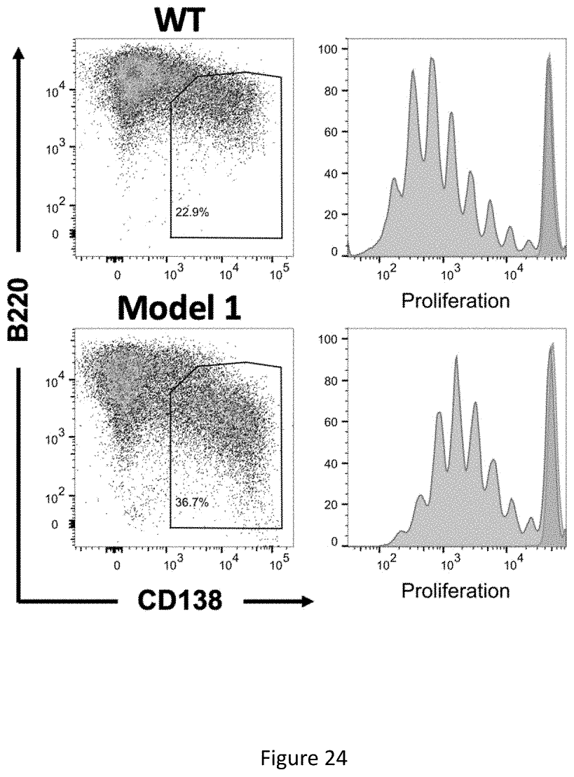

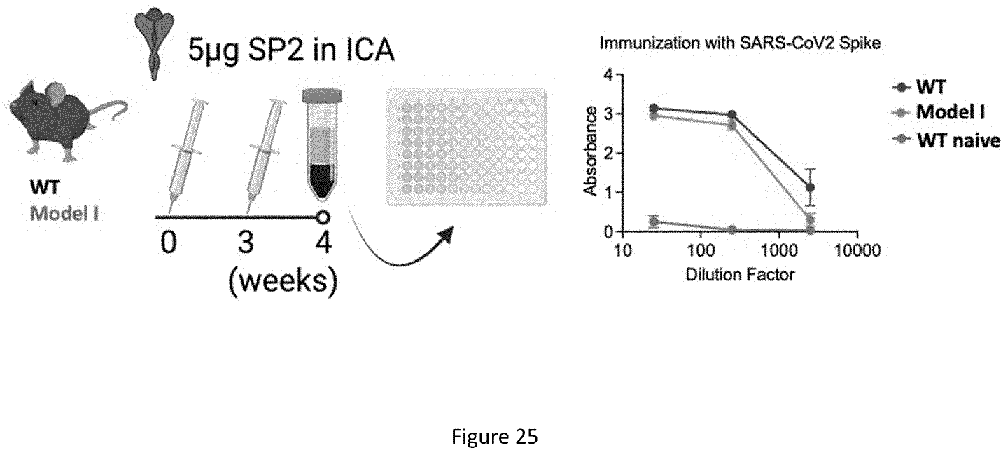

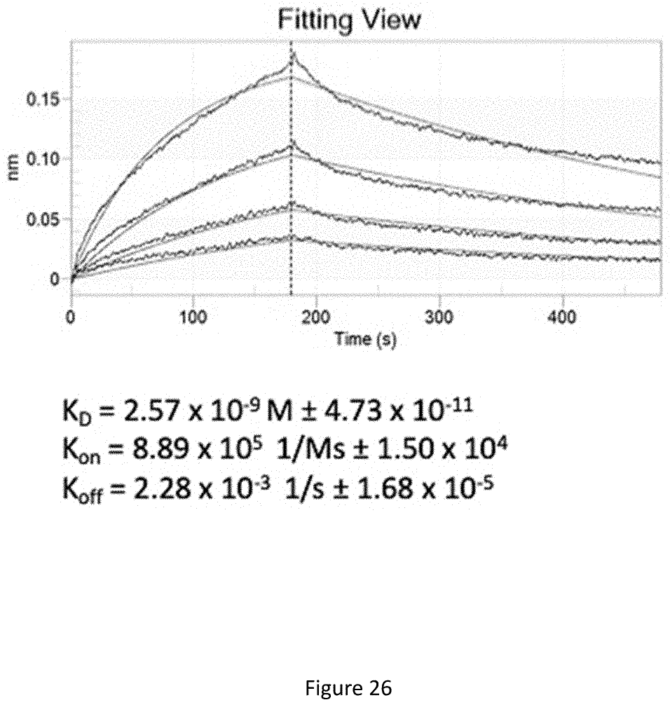

Example 6 is illustrated in FIG. 19 and FIGS. 21-26 which relate to Model I.

In an embodiment referred to herein as Model I, the disclosure provides a genetically modified non-human animal, such as a modified mouse. The genome of the modified non-human animal comprises: at least one unarranged immunoglobulin heavy chain variable domain, at least one unarranged immunoglobulin heavy chain D domain, and one unarranged immunoglobulin heavy chain J domain are operably linked to a functional non-human immunoglobulin heavy chain constant gene sequence, wherein the CH1 domain from constant region is deleted, enabling expression of a single immunoglobulin heavy chain from a B cell.

For Model I, design of the guide RNAs were such the borders of the deletion and replacement with the marker cassette resulted in deletion of mouse IgM, IgD and IgG3 CH1, while leaving CH2 and CH3 intact. A pair of Cas9 guides at the 5′ end of the targeted locus (gRNA1: GTCTTTTGAGTACCGTTGTCTGG (SEQ ID NO:118) gRNA2: CCAGCAGGTCGGCTGGACTAACT (SEQ ID NO:119)) and a pair of guides at the 3′ end of the locus (gRNA3: ATCGGTGAGAGGGTAACTAAGGG (SEQ ID NO:120) gRNA4: ACCTGTCAATGATCATATCCAGG (SEQ ID NO:121)) were used in this example. Also left intact are Adam6, the IgG3 hinge, and the Emu enhancer. The resulting modified locus generates HCAbs due to the deletion of CH1, mimicking camelid CH1 skipping, which prevents use of the light chain during antibody assembly. Production of conventional antibody IgM and IgD antibodies is ablated due to deletion of these constant regions. Thus, modified mice according to Model I produce a broad repertoire of HCAbs that utilize murine VH, DH and JH elements and that do not pair require pairing with Ig light chain to be expressed on the surface of B lymphocytes or be secreted in response to antigenic challenge.

The repertoire diversity was determined using high throughput sequencing following FACS sorting of naïve mature (B220+ CD19+ CD43−) B cells and using 10×Chromium based pipeline to make libraries for Illumina sequencing.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

What is claimed is:

1. A genetically modified non-human animal, wherein the genome of the non-human animal comprises: at least one unarranged camelid VHH, at least one human immunoglobulin heavy chain D domain, and one human immunoglobulin heavy chain J domain are operably linked to a functional non-human immunoglobulin heavy chain constant gene sequence.

2. The non-human animal of claim 1, wherein the non-human animal expresses mRNA that codes a protein that is at least 70% sequence identical to a mature VHH from alpaca, camel, llama, vicunas, or guanacos.

3. The non-human animal of claim 1, wherein the camelid VHH and human immunoglobulin heavy chain D and J gene segments are capable of rearranging to form a rearranged immunoglobulin heavy chain VDJ sequence.

4. The non-human animal of claim 1, wherein the animal expresses an immunoglobulin heavy chain receptor comprising at least one camelid VHH variable region on the surface of a B cells.

5. The non-human animal of claim 1, wherein the animal is heterozygous.

6. A method comprising immunizing a non-human animal of claim 1; separating a heavy chain only antibody from the non-human animal, and determining a sequence of one more Complementarity-determining regions (CDRs) of the separating a heavy chain only antibody, and wherein the CDRs include at least CDR3.

7. The non-human animal of claim 1, wherein the non-human animal is a rodent.

8. The non-human animal of claim 1, wherein the non-human animal is a mouse.

9. The non-human animal of claim 1, wherein the camelid VHH are at least 70% identical to human Vγ and Vδ.

10. The non-human animal of claim 1, wherein the immunoglobulin heavy chain VHH domain is at least 70% identical to one of camelid VHH.

11. The non-human animal of claim 1, wherein the immunoglobulin heavy chain D or J are at least 70% identical to human immunoglobulin heavy chain D or J.

12. The a non-human animal of claim 1, wherein the immunoglobulin heavy chain further comprise of a human Emu.

13. A genetically modified non-human animal, wherein the genome of the non-human animal comprises: at least one unarranged camelid VHH, at least one human immunoglobulin heavy chain D domain, and one human immunoglobulin heavy chain J domain either inserted into the endogenous immunoglobulin heavy chain locus or replaced at least one nucleotide or replaced at least one of the endogenous V, D, and J, wherein the unarranged segments are operably linked to a functional non-human immunoglobulin heavy chain constant gene sequence.

14. The non-human animal of claim 13, wherein the non-human animal expresses mRNA that encodes a protein that is at least 70% sequence identical to a mature VHH from alpaca, camel, llama, vicunas, or guanacos.

15. The non-human animal of claim 13, wherein the camelid VHH and human immunoglobulin heavy chain D and J gene segments are capable of rearranging to form a rearranged immunoglobulin heavy chain VDJ sequence.

16. The non-human animal of claim 13, wherein non-human animal expresses an immunoglobulin heavy chain receptor comprising at least one camelid VHH variable region on the surface of a B cells.

17. The non-human animal of claim 13, wherein the animal is heterozygous.

18. A method comprising immunizing a non-human animal of claim 12; separating a heavy chain only antibody from the non-human animal, and determining a sequence of one more Complementarity-determining regions (CDRs) of the heavy chain only antibody, and wherein the CDRs include at least CDR3.

19. The non-human animal of claim 13, wherein the animal is a rodent.

20. The non-human animal of claim 13, wherein the animal is a mouse.

21. The non-human animal of claim 20, wherein the mouse is Mus musculus.

22. The non-human animal of claim 13, wherein the immunoglobulin heavy chain VHH domain is at least 70% identical to one of camelid VHH or shark VH.

23. The non-human animal of claim 13, wherein the immunoglobulin heavy chain D or J are at least 70% identical to human immunoglobulin heavy chain D or J.

24. The non-human animal of claim 13, wherein the immunoglobulin heavy chain locus further sequence includes human Emu enhancer.

25. A method of making a genetically modified non-human animal that expresses a B cell receptor comprising camelid VHH, immunoglobulin heavy chain D and J, operably linked to a functional non-human immunoglobulin heavy chain constant region by replacing endogenous sequences with corresponding camelid-human chimeric sequences.

26. A genetically modified rodent, wherein the genome of the genetically modified rodent comprises: at least one unarranged camelid VHH, at least one human immunoglobulin heavy chain D domain, and one human immunoglobulin heavy chain J domain are operably linked to a functional non-human immunoglobulin heavy chain constant gene sequence.

27. The genetically modified rodent of claim 26, wherein the genetically modified rodent expresses mRNA that codes a protein that is at least 70% sequence identical to a mature VHH from alpaca, camel, llama, vicunas, or guanacos.

28. The genetically modified rodent of claim 26, wherein the camelid VHH and human immunoglobulin heavy chain D and J gene segments are capable of rearranging to form a rearranged immunoglobulin heavy chain VDJ sequence.

29. The genetically modified rodent of claim 26, wherein the genetically modified rodent expresses an immunoglobulin heavy chain receptor comprising at least one camelid VHH variable region on the surface of a B cells.

30. The genetically modified rodent of claim 26, wherein the rodent is heterozygous.

31. A method comprising immunizing a non-human animal of claim 26; separating a heavy chain only antibody from the non-human animal, and determining a sequence of one more Complementarity-determining regions (CDRs) of the heavy chain only antibody, and wherein the CDRs include at least CDR3.

32. The rodent of claim 26, wherein the rodent is a mouse.

33. The rodent of claim 26, wherein rodent is Mus musculus.

34. The rodent of claim 26, wherein the immunoglobulin heavy chain VHH domain is at least 70% identical to one of camelid VHH.

35. The rodent of claim 26, wherein the immunoglobulin heavy chain D or J are at least 70% identical to human immunoglobulin heavy chain D or J.

36. The rodent of claim 26, wherein the immunoglobulin heavy chain locus sequence includes human Emu enhancer.

37. A genetically modified rodent, wherein the genome of the genetically modified rodent comprises: at least one unarranged camelid VHH, at least one human immunoglobulin heavy chain D domain, and one human immunoglobulin heavy chain J domain either inserted into the endogenous immunoglobulin heavy chain locus or replaced at least one nucleotide or replaced at least one of the endogenous V, D, and J, wherein the unarranged human segments are operably linked to a functional non-human immunoglobulin heavy chain constant gene sequence.

38. The genetically modified rodent of claim 37, wherein the rodent expresses mRNA that encodes a protein that is at least 70% sequence identical to a mature VHH from alpaca, camel, llama, vicunas, or guanacos.

39. The genetically modified rodent of claim 37, wherein the camelid VHH and human immunoglobulin heavy chain D and J gene segments are capable of rearranging to form a rearranged immunoglobulin heavy chain VDJ sequence.

40. The genetically modified rodent of claim 37, wherein the genetically modified rodent expresses an immunoglobulin heavy chain receptor comprising at least one camelid VHH variable region on the surface of a B cells.

41. The genetically modified rodent of claim 37, wherein the rodent is heterozygous.

42. A method comprising immunizing a genetically modified rodent of claim 37; separating a heavy chain only antibody from the non-human animal, and determining a sequence of one more Complementarity-determining regions (CDRs) of the heavy chain only antibody, and wherein the CDRs include at least CDR3. 43. The genetically modified rodent t of claim 37, wherein the animal is a rodent.

43. The genetically modified rodent of claim 37, wherein the animal is a mouse.

44. The genetically modified rodent of claim 43, wherein the mouse is Mus musculus.

45. The genetically modified rodent of claim 37, wherein the immunoglobulin heavy chain VHH domain is at least 70% identical to one of camelid VHH.

46. The genetically modified rodent of claim 37, wherein the immunoglobulin heavy chain D or J are at least 70% identical to human immunoglobulin heavy chain D or J.

47. A method of making a genetically modified rodent that expresses a B cell receptor comprising camelid VHH, immunoglobulin heavy chain D and J, operably linked to a functional non-human immunoglobulin heavy chain constant region by replacing endogenous sequences with corresponding camelid-human chimeric sequences.

48. The genetically modified rodent of claim 47, wherein the immunoglobulin heavy chain further comprise a human Emu.

49. A genetically modified non-human animal, wherein the genome of the animal comprises: at least one unarranged immunoglobulin heavy chain variable domain, at least one unarranged immunoglobulin heavy chain D domain, and one unarranged immunoglobulin heavy chain J domain are operably linked to a functional non-human immunoglobulin heavy chain constant gene sequence, wherein the CH1 domain from constant region is deleted, enabling expression of single immunoglobulin heavy chain on a B cell.

50. The genetically modified non-human animal of claim 49, wherein the animal expresses mRNA that codes a repertoire of heavy chains capable of single-chain antibody (HCAb) production independent of light chain from the endogenous locus by virtue of modified constant region.

51. The genetically modified non-human animal of claim 50, wherein the endogenous immunoglobulin heavy chain D and J gene segments are capable of rearranging to form a rearranged immunoglobulin heavy chain VDJ sequence.

52. The genetically modified non-human animal of claim 50, wherein the animal is heterozygous.

53. A method comprising immunizing a genetically modified non-human animal of claim 50 and isolating one or more single-hain antibodies from the genetically modified non-human and determining the sequence of at least CDR3 of the one or more single-chain antibodies.

54. The genetically modified non-human animal of claim 50, wherein the animal is a rodent.

55. The genetically modified non-human animal of claim 50, wherein the animal is a mouse.

56. The genetically modified non-human animal of claim 50, wherein the genetically modified non-human animal is a rodent.

57. The genetically modified non-human animal of claim 50, wherein the genetically modified non-human animal is a mouse with an IgH locus lacking IgM and IgD constant regions.

58. The genetically modified non-human animal of claim 50, wherein the genetically modified non-human animal is a mouse with a CH1 exon(s) of IgG constant region(s) is ablated.


SEQUENCE LISTING (121 sequences)

SEQ ID NO: 1 - DNA, 350 bp, synthetic construct
  source 1..350: mol_type = other DNA
atggaactggggctgagctgggtcgtcctggctgctcttctacaaggtgtccaagctgaggtgcagctggtggagtccgggggaggctcggtgcaggctggagggtctctgagactctcctgtgcagcttctggatacacctacagtagctgcagcatgggctggtaccgccaagctccaggaaaggagcgcgaattggtctcaactattattagtgatggtagcacacactatgcagattccatgaagggccgattcacaatctccagagacaatgccaagaacgtgctgtatctgcaaatgaacagtctgaaacctgaggatacggccatgtattactgtactagaca

SEQ ID NO: 2 - DNA, 350 bp, synthetic construct
  source 1..350: mol_type = other DNA
atggagctcggactgagctgggtcgtcctggctgctcttctacaaggtgtccaggcacaggtgcagctggtcgagtctggaggagggttggtgcagcctggagggtctctgaggctctcctgtgcagcctccggaagcatctttagtatcaatgccatggggtggtaccgccaggcaccaggaaagcagcgcgagttagtcgcagctattactagcggtggtagcacaaattatgcagactccgtgaaaggccgatttaccatctctagagacaacgcaaagaatacggtgtacctgcagatgaatagcctcaaacctgaggacacagccgtctactattgtaatgcaga

SEQ ID NO: 3 - DNA, 350 bp, synthetic construct
  source 1..350: mol_type = other DNA
atggagctggggctgagctgggtggtcctggctgctctactacaaggtgtccaggcccaggtgcagctggtggagtctgggggaggcttggtgcaggctgggggctctctgagacactcctgtgcagcctctggactcaccttcggtagctatgccatgggctggtaccgccaggctccagggaaggagcgcgagttggtcgcagctattagtagtggtggtagcacatactatgcagactctgtgaagggccaattcaccatctccagagacaacgccaagaacacgctgtatctgcaaatgaacagcctgaaacctggggacacggccgtgtattactgtgcaaaaga

SEQ ID NO: 4 - DNA, 353 bp, synthetic construct
  source 1..353: mol_type = other DNA
atggagttgggtctttctctggtggtccttgccgctttgcttcagggtgtactggccgaggtgcaactcgtagaaagtggtgggggtctcgttcagcctgggggctccttgcgactttcttgcgccgcatcaggcttcaccctggattattatagtatcggctggtttcgacaagcacctgggaaagagcgagagggtgtcagttgtatcagcgactccgatgggagaacctactatgctgatagtgtaaaaggtcgattcaccatatctcgggataatgcaaaaaatactgtatatctccagatgaactctcttaaacccgaggacactgctgtttattattgtgcaacaga

SEQ ID NO: 5 - DNA, 39 bp, synthetic construct
  source 1..39: mol_type = other DNA
acatacaaattaaagtaatatgaagctctgtcctgtacc

SEQ ID NO: 6 - DNA, 119 bp, synthetic construct
  source 1..119: mol_type = other DNA
gggtaatatagaaaggcaggccaaagaacctccgaggtttcaaccaaaagcaattgtgtacaccagccaggacagaaatgcctcgacttcgctgctgcccaaggttgccgggtgacgca

SEQ ID NO: 7 - DNA, 167 bp, synthetic construct
  source 1..167: mol_type = other DNA
agatcagttggaagaatttgtccactacgtgaaaggcgagatcaccaaggtagtcggcaaataaggtacaactagtgggcccgatatccccgggtattgccggcggtaccataacttcgtataatgtatgctatacgaagttatcggataacactcaggtaggtggt

SEQ ID NO: 8 - DNA, 177 bp, synthetic construct
  source 1..177: mol_type = other DNA
gagggcaaaggaataggcgggactctggggttcgaataaagaccgaccaagcgacgtctgagagctccctgtaactttaaataattggcattatttaaagttaggatccttgccggctagcactgcaggcatgtctaggcaactccctgtgcactatgaccctggggcgttggagat

SEQ ID NO: 9 - DNA, 138 bp, synthetic construct
  source 1..138: mol_type = other DNA
gaggagcccagcactagaagtcggcggtgtttccattcggtgatcagcactgaacacagaggactcaccatggagttgggtctttctctggtggtccttgccgctttgcttcaggtaattcatggagaacaagagctt

SEQ ID NO: 10 - DNA, 513 bp, synthetic construct
  source 1..513: mol_type = other DNA
tctcttaaacccgaggacactgctgtttattattgtgcaacagagacacagtgaggggaagtcattgtgcgcccagacacaaacctccctgcaggaacgctggcgggaaatcagcggcagggggcgctcaggagccactgatcagagtcagccctggaggcaggtgcagatggaggctgtttcctgtcaggatgtgggactttgtcttcttctgacagttccccagggaacctcttaaatttagaaaactgtgcctaacaatgtcttctctatgcatatgaggaccttttctccctggcacaaaatgcagattgacgctgacacggatgaaaattcctcaaccatggtcacaaggatcagagtcctgagtaacctcagggcttcctggtgagtcttctccaatcagacccaggacagggacctccgtgagattccctgactgctagcggcttctctgagatcagccctggggggctgtgttctaggtcatgtctctcactttccactatgaga

SEQ ID NO: 11 - DNA, 60 bp, synthetic construct
  source 1..60: mol_type = other DNA
cacccaacaaccacatccctcctcagaagcccccagagcacaacgcctcaccatggagct

SEQ ID NO: 12 - DNA, 130 bp, synthetic construct
  source 1..130: mol_type = other DNA
acctgcagatgaatagcctcaaacctgaggacacagccgtctactattgtaatgcagacacagtgtgaaaacccacatcctgagagtgtcagaaaccccaggggggaagcagctgtgctggcatggagga

SEQ ID NO: 13 - DNA, 138 bp, synthetic construct
  source 1..138: mol_type = other DNA
tgccctgagagcatcacccagcaaccacatctgtcctctagagaatcccctgagagctccgttcctcaccatggagctggggctgagctgggtggtcctggctgctctactacaaggtgagtgctgtggtcagggact

SEQ ID NO: 14 - DNA, 120 bp, synthetic construct
  source 1..120: mol_type = other DNA
tgtatctgcaaatgaacagcctgaaacctggggacacggccgtgtattactgtgcaaaagacacagtgtgaaaacccacatcctgagggtgtcagaaaccccagggaggaggcagctgtg

SEQ ID NO: 15 - DNA, 119 bp, synthetic construct
  source 1..119: mol_type = other DNA
gaactgcagagcctgctgaattctggctgaccagggcagtcaccagagctccagacaatggaactggggctgagctgggtcgtcctggctgctcttctacaaggtgagtgtctcaggaa

SEQ ID NO: 16 - DNA, 115 bp, synthetic construct
  source 1..115: mol_type = other DNA
atctgcaaatgaacagtctgaaacctgaggatacggccatgtattactgtactagacacacagtgaggggaagtcagtgtgagcccagacacaaacctccctgcagggatgctca

SEQ ID NO: 17 - DNA, 156 bp, synthetic construct
  source 1..156: mol_type = other DNA
ccagcccccatccaggaggccccagagctcagggcgccggggcagattctgaacagccccgagtcacggtgggtacaactggaacgaccaccgtgagaaaaactgtgtccaaaactctctcctggcccctgctggaggccgcgccagagaggggag

SEQ ID NO: 18 - DNA, 206 bp, synthetic construct
  source 1..206: mol_type = other DNA
gcccggagacagaaggtctctgggtggctgggtttttgtggggtgaggatggacattctgccattgtgattactactactactactacatggacgtctggggcaaagggaccacggtcaccgtctcctcaggtaagaatggccactctagggcctttgttttctgctactgcctgtggggtttcctgagcattgcaggttggtcct

SEQ ID NO: 19 - DNA, 185 bp, synthetic construct
  source 1..185: mol_type = other DNA
ggagactcatttatgtgagtcttttgagtgaccattgtctgggtcactcccatttaactttccctcgtacgttcgtgggattgtgtccgtgtcgcgaagttcctatactttctagagaataggaacttcccgcggttgtaagttctccagatctagagtaagacagaagtctggaagacatggga

SEQ ID NO: 20 - DNA, 168 bp, synthetic construct
  source 1..168: mol_type = other DNA
ccgcatgctccagactgccttgggaaaagcgcctcccctacccggtagaatgaagttcctatactttctagagaataggaacttcgttggtaccgtacggcaatgccaatcagggcagttgggttcctttgtgtctgcctcctagaagcctcttttcatgttcctcat

SEQ ID NO: 21 - DNA, 67 bp, synthetic construct
  source 1..67: mol_type = other DNA
gaagctgagatattaggctttaacgagggccaagcaataacactatccctgaccacatggtaaggct

SEQ ID NO: 22 - DNA, 25 bp, Mus musculus
  source 1..25: mol_type = other DNA
tccagtcctggtgctggatggggag

SEQ ID NO: 23 - DNA, 27 bp, Lama pacos
  source 1..27: mol_type = other DNA
atggagctcggactgagctgggtcgtc

SEQ ID NO: 24 - DNA, 28 bp, Camelus dromedarius
  source 1..28: mol_type = other DNA
atggaactggggctgagctgggtcgtcc

SEQ ID NO: 25 - DNA, 28 bp, Lama glama
  source 1..28: mol_type = other DNA
atggagctggggctgagctgggtggtcc

SEQ ID NO: 26 - DNA, 30 bp, Lama glama
  source 1..30: mol_type = other DNA
tggagttgggtctttctctggtggtccttg

SEQ ID NO: 27 - DNA, 26 bp, Mus musculus
  source 1..26: mol_type = other DNA
gaaggtactgttgtactgagcttcac

SEQ ID NO: 28 - DNA, 28 bp, Lama pacos
  source 1..28: mol_type = other DNA
tgcagcctggagggtctctgaggctctc

SEQ ID NO: 29 - DNA, 29 bp, Camelus dromedarius
  source 1..29: mol_type = other DNA
agactctcctgtgcagcttctggatacac

SEQ ID NO: 30 - DNA, 28 bp, Lama glama
  source 1..28: mol_type = other DNA
gacactcctgtgcagcctctggactcac

SEQ ID NO: 31 - DNA, 26 bp, Lama glama
  source 1..26: mol_type = other DNA
ggccgaggtgcaactcgtagaaagtg

SEQ ID NO: 32 - DNA, 351 bp, synthetic construct
  source 1..351: mol_type = other DNA
aggtgcagctggtcgagtctggaggagggttggtgcagcctggagggtctctgaggctctcctgtgcagcctccggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaaggagtgggagctgatgcttttgatatctggggccaagggacaatggtcaccgtctcttcag

SEQ ID NO: 33 - DNA, 357 bp, synthetic construct
  source 1..357: mol_type = other DNA
aggtgcagctggtcgagtctggaggagggttggtgcagcctggagggtctctgaggctctcctgtgcagcctccggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaagatgcgagcagcagctggccgccttactacatggacgtctggggcaaagggaccacggtcaccgtct

SEQ ID NO: 34 - DNA, 354 bp, synthetic construct
  source 1..354: mol_type = other DNA
aggtgcagctggtcgagtctggaggagggttggtgcagcctggagggtctctgaggctctcctgtgcagcctccggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaagttgatatagtggacaactggttcgacccctggggccagggaaccctggtcaccgtctcctcag

SEQ ID NO: 35 - DNA, 360 bp, synthetic construct
  source 1..360: mol_type = other DNA
aggtgcagctggtcgagtctggaggaggctcggtgcaggctggagggtctctgagactctcctgtgcagcttctggatacacctacagtagctgcagcatgggctggtaccgccaagctccaggaaaggagcgcgaattggtctcaactattattagtgatggtagcacacactatgcagattccatgaagggccgattcacaatctccagagacaatgccaagaacgtgctgtatctgcaaatgaacagtctgaaacctgaggatacggccatgtattactgtactagactaagtattactatggttcggggagcccccctctactactactactacatggacgtctggggcaaaggga

SEQ ID NO: 36 - DNA, 351 bp, synthetic construct
  source 1..351: mol_type = other DNA
aggtgcagctggtcgagtctggaggagggttggtgcagcctggagggtctctgaggctctcctgtgcagcctccggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcagcagcagctggtacagatgcttttgatatctggggccaagggacaatggtcaccgtctcttcag

SEQ ID NO: 37 - DNA, 351 bp, synthetic construct; identical to SEQ ID NO: 36
  source 1..351: mol_type = other DNA

SEQ ID NO: 38 - DNA, 357 bp, synthetic construct
  source 1..357: mol_type = other DNA
aggtgcagctggtcgagtctggaggagggttggtgcagcctggagggtctctgaggctctcctgtgcagcctccggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaagggaggtatagcagcagctggtactttgactactggggccagggaaccctggtcaccgtctcctcag

SEQ ID NO: 39 - DNA, 357 bp, synthetic construct
  source 1..357: mol_type = other DNA
aggtgcagctggtcgagtctggaggagggttggtgcagcctggagggtctctgaggctctcctgtgcagcctccggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaaagactaactggaacttatgatgcttttgatatctggggccaagggacaatggtcaccgtctcttcag

SEQ ID NO: 40 - DNA, 353 bp, synthetic construct
  source 1..353: mol_type = other DNA
aggtgcagctggtcgagtctggaggagggttggtgcagcctggagggtctctgaggctctcctgtgcagcctccggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaagatcaagactggaactttcctttgactactggggccagggaaccctggtcaccgtctcctcag

SEQ ID NO: 41 - DNA, 349 bp, synthetic construct
  source 1..349: mol_type = other DNA
gaggtgcagctggtggagtccgggggaggctcggtgcaggctggagggtctctgagactctcctgtgcagcttctggatacacctacagtagctgcagcatgggctggtaccgccaagctccaggaaaggagcgcgaattggtctcaactattattagtgatggtagcacacactatgcagattccatgaagggccgattcacaatctccagagacaatgccaagaacgtgctgtatctgcaaatgaacagtctgaaacctgaggatacggccatgtattactgtactttactacggtgtcctctctttgactactggggccagggaaccctggtcaccgtctcctcag

SEQ ID NO: 42 - DNA, 354 bp, synthetic construct
  source 1..354: mol_type = other DNA
aggtgcagctggtcgagtctggaggagggttggtgcagcctggagggtctctgaggctctcctgtgcagcctccggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgatatagtggctacgattccaggggcttttgatatctggggccaagggacaatggtcaccgtctcttcag

SEQ ID NO: 43 - DNA, 351 bp, synthetic construct
  source 1..351: mol_type = other DNA
aggtgcagctgatcgagtctggaggagggttggtgcagcctggagggtctctgaggctctcctgtgcagcctccggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaagggggctggggatctcttctttgactactggggccagggaaccctggtcaccgtctcctcag

SEQ ID NO: 44 - DNA, 357 bp, synthetic construct
  source 1..357: mol_type = other DNA
aggtgcagctggtcgagtctggaggagggttggtgcagcctggagggtctctgaggctctcctgtgcagcctccggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaagtagagtgggagctactggactggttcgacccctggggccagggaaccctggtcaccgtctcctcag

SEQ ID NO: 45 - DNA, 356 bp, synthetic construct
  source 1..356: mol_type = other DNA
aggtgtccaggcacaggtgcagctggtcgagtctggaggagggttggtgcagcctggagggtctctgaggctctcctgtgcagcctccggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcccccgtaactggctttgactactggggccagggaaccctggtcaccgtctcctcag

SEQ ID NO: 46 - DNA, 390 bp, synthetic construct
  source 1..390: mol_type = other DNA
gaggtgcaactcgtagaaagtggtgggggtctcgttcagcctgggggctccttgcgactttcttgcgccgcatcaggcttcaccctggattattatagtatcggctggttttgacaagcacctgggaaagagcgagagggtgtcagttgtatcagcgactccgatgggagaacctactatgctgatagtgtaaaaggtcgattcaccatatctcgggataatgcaaaaaatactgtatatctccagatgaactctcttaaacccgaggacactgctgtttattattgtgcaacagagaagacgatttttggagtggttattatacaacttactactactactactacatggacgtctggggcaaagggaccacggtcaccgtctcctcag

SEQ ID NO: 47 - DNA, 372 bp, synthetic construct
  source 1..372: mol_type = other DNA
gaggtgcaactcgtagaaagtggtgggggtctcgttcagcctgggggctccttgcgactttcttgcgccgcatcaggcttcaccctggattattatagtatcggctggttttgacaagcacctgggaaagagcgagagggtgtcagttgtatcagcgactccgatgggagaacctactatgctgatagtgtaaaaggtcgattcaccatatctcgggataatgcaaaaaatactgtatatctccagatgaactctcttaaacccgaggacactgctgtttattattgtgcaacaccttccatagcagctcgtcctactactactactacatggacgtctggggcaaagggaccacggtcaccgtctcctcag

SEQ ID NO: 48 - DNA, 386 bp, synthetic construct
  source 1..386: mol_type = other DNA
gaggtgcaactcgtagaaagtggtgggggtctcgttcagcctgggggctccttgcgactttcttgcgccgcatcaggcttcaccctggattattatagtatcggctggttttgacaagcacctgggaaagagcgagagggtgtcagttgtatcagcgactccgatgggagaacctactatgctgatagtgtaaaaggtcgattcaccatatctcgggataatgcaaaaaatactgtatatctccagatgaactctcttaaacccgaggacactgctgtttattattgtgcaacagaggaggggtattactatggttcggggagttatggaagactactactacatggacgtctggggcaaagggaccacggtcaccgtctcctcag

SEQ ID NO: 49 - DNA, 340 bp, synthetic construct
  source 1..340: mol_type = other DNA
gaggtgcaactcgtagaaagtggtgggggtctcgttcagcctgggggctccttgcgactttcttgcgccgcatcaggcttcaccctggattattatagtatcggctggttttgacaagcacctgggaaagagcgagagggtgtcagttgtatcagcgactccgatgggagaacctactatgctgatagtgtaaaaggtcgattcaccatatctcgggataatgcaaaaaatactgtatatctccagatgaactctcttaaacccgaggacactgctgtttattattacagcacctggtacggactactggggccagggaaccctggtcaccgtctcctca

SEQ ID NO: 50 - DNA, 372 bp, synthetic construct
  source 1..372: mol_type = other DNA
gaggtgcaactcgtagaaagtggtgggggtctcgttcagcctgggggctccttgcgactttcttgcgccgcatcaggcttcaccctggattattataatatcgtctggttttgacaaacacctgagaaagaacgagagggtgtcagttgtttcagcgactccgatggaaaaacctactctattcatagtataaaaggtcgattcaccatatctcgggataatgcaaaaaatactgtatatctccagatgaactctcttaaacccgaggacactgctgtttattactgtgcaacagatattgtaatactaccagctgctatgagaactggttcgacccctggggccagggaaccctggtcaccgtctcctcag

SEQ ID NO: 51 - DNA, 348 bp, synthetic construct
  source 1..348: mol_type = other DNA
gaggtgcaactcgtagaaagtggtgggggtctcgttcagcctgggggctccttgcgactttcttgcgccgcatcaggcttcaccctggattattatagtatcggctggttttgacaagcacctgggaaagagcgagagggtgtcagttgtatcagcgactccgatgggagaacctactatgctgatagtgtaaaaggtcgattcaccatatctcgggataatgcaaaaaatactgtatatctccagatgaactctcttaaacccgaggacactgctgtttattattgtgcaactaacatctggtaccggctgttggaggactgtggccactgaggcctggtaaccctg

SEQ ID NO: 52 - DNA, 370 bp, synthetic construct
  source 1..370: mol_type = other DNA
gaggtgcaactcgtagaaagtggtgggggtctcgttcagcctgggggctccttgcgactttcttgcgccgcatcaggcttcaccctggattattatagtatcggctggttttgacaagcacctgggaaagagcgagagggtgtcagttgtatcagcgactccgatgggagaacctactatgctgatagtgtaaaaggtcgattcaccatatctcgggataatgcaaaaaatactgtatatctccagatgaactctcttaaacccgaggacactgctgtttattattgtgcaacagagactcggggagttataaactactactactacatggacgtctggggcaaagggaccacggtcaccgtctcctcag

SEQ ID NO: 53 - DNA, 367 bp, synthetic construct
  source 1..367: mol_type = other DNA
gaggtgcaactcgtagaaagtggtgggggtctcgttcagcctgggggctccttgcgactttcttgcgccgcatcaggcttcaccctggattattatagtatcggctggttttgacaagcacctgggaaagagcgagagggtgtcagttgtatcagcgactccgatgggagaacctactatgctgatagtgtaaaaggtcgattcaccatatctcgggataatgcaaaaaatactgtatatctccagatgaactctcttaaacccgaggacactgctgtttattattgtgcaacatattgtagtagtaccagctgctactggtacttcgatctctggggccgtggcaccctggtcactgtctcctcag

SEQ ID NO: 54 - DNA, 363 bp, synthetic construct
  source 1..363: mol_type = other DNA
gaggtgcaactcgtagaaagtggtgggggtctcgttcagcctgggggctccttgcgactttcttgcgccgcatcaggcttcaccctggattattatagtatcgactggttttgacaagcacctgggaaagagcgagagggtgtcagttgtatcagcgactccgatgggagaacctactatgctgatagtgtaaaaggtcgattcaccatatctcgggataatgcaaaaaatactgtatatctccagatgaactctcttaaacccgaggacactgctgtttattattgtgcaacacatataggtatagcagcagctggtacctttgactactggggccagggaaccctggtcaccgtctcctca

SEQ ID NO: 55 - DNA, 307 bp, synthetic construct
  source 1..307: mol_type = other DNA
cagcctggagggtctctgaggctctcctgtgcagcttctggatacacctacagtagctgcagcatgggctggtaccgccaagctccaggaaaggagcgcgaattggtctcaactattattagtgatggtagcacacactatgcagattccatgaagggccgattcacaatctccagagacaatgccaagaacgtgctgtatctgcaaatgaacagtctgaaacctgaggatacggccatgtattactgtactagacaactcccctttgactactggggccagggaaccctggtcaccgtctcctcag

SEQ ID NO: 56 - DNA, 326 bp, synthetic construct
  source 1..326: mol_type = other DNA
gatgcagcctggagggtctctgaggctctcctgtgcagcttctggatacacctacagtagctgcagcatgggctggtaccgccaagctccaggaaaggagcgcgaattggtctcaactattattagtgatggtagcacacactatgcagattccatgaagggccgattcacaatctccagagacaatgccaagaacgtgctgtatctgcaaatgaacagtctgaaacctgaggatacggccatgtattactgtacttgtatagcagctcgtccggatgatgcttttgatatctggggccaagggacaatggtcaccgtctcttcag

SEQ ID NO: 57 - DNA, 319 bp, synthetic construct
  source 1..319: mol_type = other DNA
cagcctggagggtctctgaggctctcctgtgcagcctccggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaagtgatggagtgggagctcccttttgactactggggccagggaaccctggtcaccgtctcctcag

SEQ ID NO: 58 - DNA, 321 bp, synthetic construct
  source 1..321: mol_type = other DNA
cagcctggagggtctctgaggctctcctgtgcagcctccggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaagtatagcagcagctgccttgactactttgactactggggccagggaaccctggtcaccgtctcctcag

SEQ ID NO: 59 - DNA, 323 bp, synthetic construct
  source 1..323: mol_type = other DNA
gatgcagcctggagggtctctgaggctctcctgtgcagcctccggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaatataactggaactaactggggatctgactactggggccagggaaccctggtcaccgtctcctcag

SEQ ID NO: 60 - DNA, 329 bp, synthetic construct
  source 1..329: mol_type = other DNA
gatgcagcctggagggtctctgaggctctcctgtgcagcttctggatacacctacagtagctgcagcatgggctggtaccgccaagctccaggaaaggagcgcgaattggtctcaactattattagtgatggtagcacacactatgcagattccatgaagggccgattcacaatctccagagacaatgccaagaacgtgctgtatctgcaaatgaacagtctgaaacctgaggatacggccatgtattactgtactagacagtgtataactggaactaagggatactttgactactggggccagggaaccctggtcaccgtctcctcag

SEQ ID NO: 61 - DNA, 290 bp, synthetic construct
  source 1..290: mol_type = other DNA
ctctggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaagttgactacggtgactaccttgactggttcgacccctggggccagggaaccctggtcaccgtctcctcag

SEQ ID NO: 62 - DNA, 300 bp, synthetic construct
  source 1..300: mol_type = other DNA
gacactcctgtgcagcctctggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaatttagcgtatagcagctccgactttgactactggggccagggaaccctggtcaccgtctcctcag

SEQ ID NO: 63 - DNA, 285 bp, synthetic construct
  source 1..285: mol_type = other DNA
gacactcctgtgcagcctctggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaactggaaggggactactggggccagggaaccctggtcaccgtctcctcag

SEQ ID NO: 64 - DNA, 285 bp, synthetic construct
  source 1..285: mol_type = other DNA
gacactcctgtgcagcctctggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaagatagtgggagctactggggccagggaaccctggtcaccgtctcctcag

SEQ ID NO: 65 - DNA, 286 bp, synthetic construct
  source 1..286: mol_type = other DNA
tgtgcagcctctggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaagatctaactggaactacggactactggggccagggaaccctggtcaccgtctcctcag

SEQ ID NO: 66 - DNA, 294 bp, synthetic construct
  source 1..294: mol_type = other DNA
gacactcctgtgcagcctctggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaacgtactggggatgcttttgatatctggggccaagggacaatggtcaccgtctcttcag

SEQ ID NO: 67 - DNA, 300 bp, synthetic construct
  source 1..300: mol_type = other DNA
gacactcctgtgcagcctctggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaagatctccgactggggaagggccctgactactggggccagggaaccctggtcaccgtctcctcag

SEQ ID NO: 68 - DNA, 312 bp, synthetic construct
  source 1..312: mol_type = other DNA
gacactcctgtgcagcctctggactcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaaaggactggaactaccctctactactactactacatggacgtctggggcaaagggaccacggtcaccgtctcctcag

SEQ ID NO: 69 - DNA, 299 bp, synthetic construct
  source 1..299: mol_type = other DNA
gacactcctgtgcagcctctggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaagatcaagactggaactttcctttgactactggggccagggaaccctggtcaccgtctcctcag

SEQ ID NO: 70 - DNA, 299 bp, synthetic construct
  source 1..299: mol_type = other DNA
gacactcctgtgcagcctctggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaagtgatggagtgggagctcccttttgactactggggccagggaaccctggtcaccgtctcctca

SEQ ID NO: 71 - DNA, 294 bp, synthetic construct
  source 1..294: mol_type = other DNA
gacactcctgtgcagcctctggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaaggtataactggaactactgactactggggccagggaaccctggtcaccgtctcctcag

SEQ ID NO: 72 - DNA, 294 bp, synthetic construct
  source 1..294: mol_type = other DNA
gacactcctgtgcagcctctggacgcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaaggcgaggactggaaccttgactactggggccagggaaccctggtcaccgtctcctcag

SEQ ID NO: 73 - DNA, 297 bp, synthetic construct
  source 1..297: mol_type = other DNA
gacactcctgtgcagcctctggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcagcagcagctggtacagatgcttttgatatctggggccaagggacaatggtcaccgtctcttcag

SEQ ID NO: 74 - DNA, 317 bp, synthetic construct
  source 1..317: mol_type = other DNA
gacactcctgtgcagcctctggaagcatctttagtatcaatgccatggggtggtaccgccaggcaccaggaaagcagcgcgagttagtcgcagctattactagcggtggtagcacatactatgcagactctgtgaagggccaattcaccatctccagagacaacgccaagaacacgctgtatctgcaaatgaacagcctgaaacctggggacacggccgtgtattactgtgcaaaagattactatggttcggggagttattataacgtgactggttcgacccctggggccagggaaccctggtcaccgtctcctcag

SEQ ID NO: 75 - protein, 11 aa, Lama pacos
AKGVGADAFDI

SEQ ID NO: 76 - protein, 15 aa, Lama pacos
AKDASSSWPPYYMDV

SEQ ID NO: 77 - protein, 12 aa, Lama pacos
AKVDIVDNWFDP

SEQ ID NO: 78 - protein, 7 aa, Lama pacos
APADFDY

SEQ ID NO: 79 - protein, 21 aa, Lama pacos
TRLSITMVRGAPLYYYYYMDV

SEQ ID NO: 80 - protein, 11 aa, Lama pacos
AAAAGTDAFDI

SEQ ID NO: 81 - protein, 13 aa, Lama pacos
AKGRYSSSWYFDY

SEQ ID NO: 82 - protein, 13 aa, Lama pacos
AKRLTGTYDAFDI

SEQ ID NO: 83 - protein, 12 aa, Lama pacos
AKDQDWNFPLTT

SEQ ID NO: 84 - protein, 10 aa, Camelus dromedarius
TLLRCPLFDY

SEQ ID NO: 85 - protein, 12 aa, Camelus dromedarius
DIVATIPGAFDI

SEQ ID NO: 86 - protein, 11 aa, Camelus dromedarius
AKGAGDLFFDY

SEQ ID NO: 87 - protein, 13 aa, Camelus dromedarius
AKVEWELLDWFDP

SEQ ID NO: 88 - protein, 8 aa, Lama glama
APVTGFDY

SEQ ID NO: 89 - protein, 23 aa, Lama glama
ATEKTIFGVVIIQLTTTTTTWTS

SEQ ID NO: 90 - protein, 17 aa, Lama glama
ATPSIAARPTTTTTWTS

SEQ ID NO: 91 - protein, 21 aa, Lama glama
ATEEGYYYGSGSYGRLLLHGR

SEQ ID NO: 92 - protein, 6 aa, Lama glama
STWYGL

SEQ ID NO: 93 - protein, 17 aa, Lama glama
ATDIVILPAAMRTGSTP

SEQ ID NO: 94 - protein, 14 aa, Lama glama
ATNIWYRLLEDCGH

SEQ ID NO: 95 - protein, 16 aa, Lama glama
ATETRGVINYYYYMDV

SEQ ID NO: 96 - protein, 15 aa, Lama glama
ATYCSSTSCYWYFDL

SEQ ID NO: 97 - protein, 14 aa, Lama glama
ATHIGIAAAGTFDY

SEQ ID NO: 98 - protein, 8 aa, Lama pacos
TRQLPFDY

SEQ ID NO: 99 - protein, 13 aa, Lama pacos
TCIAARPDDAFDI

SEQ ID NO: 100 - protein, 12 aa, Lama pacos
AKVMEWELPFDY

SEQ ID NO: 101 - protein, 13 aa, Lama pacos
ASIAAAALTTLTT

SEQ ID NO: 102 - protein, 12 aa, Lama pacos
ANITGTNWGSDY

SEQ ID NO: 103 - protein, 14 aa, Lama pacos
TRQCITGTKGYFDY

SEQ ID NO: 104 - protein, 14 aa, Lama glama
AKVDYGDYLDWFDP

SEQ ID NO: 105 - protein, 12 aa, Lama glama
ANLAYSSSDFDY

SEQ ID NO: 106 - protein, 7 aa, Lama glama
AKLEGDY

SEQ ID NO: 107 - protein, 7 aa, Lama glama
AKDSGSY

SEQ ID NO: 108 - protein, 10 aa, Lama glama
AKDLTGTTDY

SEQ ID NO: 109 - protein, 10 aa, Lama glama
AKRTGDAFDI

SEQ ID NO: 110 - protein, 12 aa, Lama glama
AKDLRLGKGPDY

SEQ ID NO: 111 - protein, 16 aa, Lama glama
AKRTGTTLYYYYYMDV

SEQ ID NO: 112 - protein, 12 aa, Lama glama
AKDQDWNFPLTT

SEQ ID NO: 113 - protein, 12 aa, Lama glama
AKVMEWELPFDY

SEQ ID NO: 114 - protein, 10 aa, Lama glama
AKGITGTTDY

SEQ ID NO: 115 - protein, 10 aa, Lama glama
AKGEDWNLDY

SEQ ID NO: 116 - protein, 11 aa, Lama glama
AAAAGTDAFDI

SEQ ID NO: 117 - protein, 18 aa, Lama glama
AKDYYGSGSYYNVTGSTP

SEQ ID NO: 118 - DNA, 23 bp, synthetic construct
  source 1..23: mol_type = other DNA
gtcttttgagtaccgttgtctgg

SEQ ID NO: 119 - DNA, 23 bp, synthetic construct
  source 1..23: mol_type = other DNA
ccagcaggtcggctggactaact

SEQ ID NO: 120 - DNA, 23 bp, synthetic construct
  source 1..23: mol_type = other DNA
atcggtgagagggtaactaaggg

SEQ ID NO: 121 - DNA, 23 bp, synthetic construct
  source 1..23: mol_type = other DNA
acctgtcaatgatcatatccagg